(19) United States
(12) Patent Application Publication
(10) Pub. No.: US 2025/0066296 A1
(43) Pub. Date: Feb. 27, 2025

(54) METHODS FOR THE SYNTHESIS OF COMPLEMENT FACTOR D INHIBITORS AND INTERMEDIATES THEREOF

(71) Applicant: Alexion Pharmaceuticals, Inc., Boston, MA (US)

(72) Inventors: Akihiro Hashimoto, Guilford, CT (US); Avinash Phadke, Cambridge, MA (US); Sankappa Rai U, Bangalore (IN); Prabu Chandran, Ooty (IN)

(73) Assignee: Alexion Pharmaceuticals, Inc., Boston, MA (US)

(21) Appl. No.: 18/719,260

(22) PCT Filed: Dec. 13, 2022
(86) PCT No.: PCT/US2022/052695

§ 371 Date: Jun. 13, 2024

Related U.S. Application Data
(60) Provisional application No. 63/289,368, filed on Dec. 14, 2021.

(51) Int. Cl. C07D 207/08; B01J 21/18; B01J 23/44; C07D 401/14; C07D 471/04; C07D 487/04

(52) U.S. Cl. CPC C07D 207/08; B01J 21/18; B01J 23/44; C07D 401/14; C07D 471/04; C07D 487/04

(57) ABSTRACT

The present disclosure provides methods for the synthesis of complement factor D inhibitors and intermediates thereof.

### BACKGROUND

The complement system is a part of the innate immune system which does not adapt to changes over the course of the host's life, but is recruited and used by the adaptive immune system. For example, it assists, or complements, the ability of antibodies and phagocytic cells to clear pathogens. This sophisticated regulatory pathway allows rapid reaction to pathogenic organisms while protecting host cells from destruction. Over thirty proteins and protein fragments make up the complement system. These proteins act through opsonization (enhancing phagocytosis of antigens), chemotaxis (attracting macrophages and neutrophils), cell lysis (rupturing membranes of foreign cells), and agglutination (clustering and binding of pathogens together).

The complement system has three pathways: classical, alternative, and lectin. Complement Factor D plays an early and central role in activation of the alternative pathway of the complement cascade. Activation of the alternative complement pathway is initiated by spontaneous hydrolysis of a thioester bond within the C3 protein to produce C3(H₂O), which associates with Factor B to form the C3(H₂O)B complex. Complement Factor D acts to cleave Factor B within the C3(H₂O)B complex to form Ba and Bb. The Bb fragment remains associated with C3(H₂O) to form the alternative pathway C3 convertase C3(H₂O)Bb. Additionally, C3b generated by any of the C3 convertases also associates with Factor B to form C3bB, which Factor D cleaves to generate the later stage alternative pathway C3 convertase C3bBb. This latter form of the alternative pathway C3 convertase may provide important downstream amplification within all three of the defined complement pathways, leading ultimately to the recruitment and assembly of additional factors in the complement cascade pathway, including the cleavage of C5 to C5a and C5b. C5b acts in the assembly of factors C6, C7, C8, and C9 into the membrane attack complex, which can destroy pathogenic cells by lysing the cell.

The dysfunction of or excessive activation of complement has been linked to certain autoimmune, inflammatory, and neurodegenerative diseases, as well as ischemia-reperfusion injury and cancer. For example, activation of the alternative pathway of the complement cascade contributes to the production of C3a and C5a, both potent anaphylatoxins, which also have roles in a number of inflammatory disorders. Therefore, in some instances, it is desirable to decrease the response of the complement pathway, including the alternative complement pathway. Some examples of disorders mediated by the complement pathway include age-related macular degeneration (AMD), paroxysmal nocturnal hemoglobinuria (PNH), multiple sclerosis, and rheumatoid arthritis.

Additional complement-mediated disorders include those classified under component 3 glomerulopathy (C3G). C3G is a recently defined entity comprised of dense deposit disease (DDD) and C3 glomerulonephritis (C3GN) which encompasses a population of chronic kidney diseases wherein elevated activity of the alternative complement pathway and terminal complement pathway results in glomerular deposits made solely of complement C3 and no immunoglobulin (Ig).

Immune-complex membranoproliferative glomerulonephritis (IC-MPGN) is a renal disease which shares many clinical, pathologic, genetic and laboratory features with C3G, and therefore can be considered a sister disease of C3G. In the majority of patients with IC-MPGN, an underlying disease or disorder—most commonly infections, autoimmune diseases, or monoclonal gammopathies—are identified to which the renal disease is secondary. Patients with idiopathic IC-MPGN can have low C3 and normal C4 levels, similar to those observed in C3G, as well as many of the same genetic or acquired factors that are associated with abnormal alternative pathway activity. Although there are current hypotheses suggesting that the majority of IC-MPGN is attributable to over activity of the classical pathway, those patients with a low C3 and a normal C4 are likely to have significant overactivity of the alternative pathway. IC-MPGN patients with a low C3 and a normal C4 may benefit from alternative pathway inhibition.

Other disorders that have been linked to the complement cascade include atypical hemolytic uremic syndrome (aHUS), hemolytic uremic syndrome (HUS), abdominal aortic aneurysm, hemodialysis complications, hemolytic anemia, or hemodialysis, neuromyelitis optica (NMO), myasthenia gravis (MG), fatty liver, nonalcoholic steatohepatitis (NASH), liver inflammation, cirrhosis, liver failure, dermatomyositis, and amyotrophic lateral sclerosis.

Factor D is an attractive target for inhibition or regulation of the complement cascade due to its early and essential role in the alternative complement pathway, and for its potential role in signal amplification within the classical and lectin complement pathways. Inhibition of Factor D effectively interrupts the pathway and attenuates the formation of the membrane attack complex.

To this end, a number of small molecule Factor D inhibitors have been developed and investigated for potential therapeutic uses. Examples of these Factor D inhibiting compounds methods of preparing them are described in PCT patent publications WO2015/130838, WO2017/035353, WO2017/035409, WO2018/160891, and WO2018/160892.

New methods for the synthesis of small molecule Factor D inhibitors and intermediates thereof are desirable.

### SUMMARY OF THE DISCLOSURE

The present disclosure generally relates to an improved method of preparing compounds useful for treating disorders mediated by complement factor D and intermediates thereof.

In particular, the present disclosure provides method of preparing a compound of Formula (VIII):

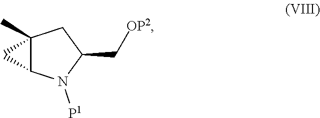

in which P¹ is H or an N-protecting group and P² is H or a hydroxyl protecting group. The method includes providing a compound of formula (VII):

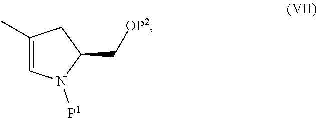

in which P¹ is H or an N-protecting group and P² is H or a hydroxyl protecting group (e.g., an ester hydroxyl protecting group such as benzoyl) and forming the compound of formula (VIII) from the compound of formula (VII), said forming the compound of formula (VII) comprising reacting the compound of formula (II) under Simmons-Smith reaction conditions.

In some embodiments, said reacting the compound of formula (VII) under Simmons-Smith reaction conditions comprises reacting the compound of formula (VII) with diethylzinc and chloroiodomethane. The reaction is typically performed in an organic solvent, e.g., toluene or methylene chloride.

In some embodiments, said providing the compound of formula (VII) includes providing a compound of formula (VI):

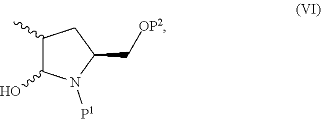

in which P¹ is an N-protecting group and P² is a hydroxyl protecting group (e.g., an ester hydroxyl protecting group such as benzoyl) and subjecting the compound of formula (VI) to a dehydration reaction. The dehydration reaction may include reacting the compound of formula (VI) with trifluoroacetic anhydride in the presence of 2,6-lutidene.

In some embodiments, said providing the compound of formula (VI) includes providing a compound of formula (V):

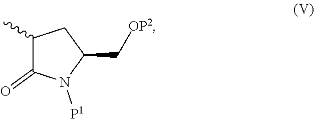

in which P¹ is an N-protecting group and P² is a hydroxyl protecting group (e.g., an ester hydroxyl protecting group such as benzoyl) and reacting the compound of formula (V) with a reducing agent, e.g., super hydride.

In some embodiments, said providing the compound of formula (V) includes providing a compound of formula (IV):

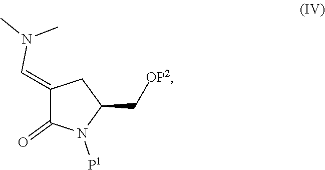

in which P¹ is H or an N-protecting group and P² is H or a hydroxyl protecting group (e.g., an ester hydroxyl protecting group such as benzoyl) and subjecting the compound of formula (IV) to a hydrogenolysis reaction in the presence of a hydrogenation catalyst, e.g., palladium on carbon (Pd/C).

In some embodiments, said providing the compound of formula (IV) includes providing a compound of formula (III):

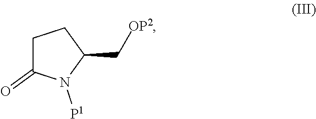

in which P¹ is an N-protecting group and P² is a hydroxyl protecting group (e.g., an ester hydroxyl protecting group such as benzoyl) and reacting the compound of formula (III) with Bredereck's reagent.

In some embodiments, said providing the compound of formula (III) includes providing a compound of formula (IIA):

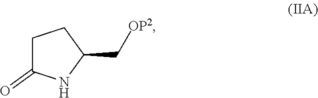

in which P² is a hydroxyl protecting group and reacting the compound of formula (IIA) with an N-protecting reagent. In some embodiments, said providing the compound of formula (IIA) comprises reacting (S)-5-(hydroxymethyl)pyrrolidin-2-one with a hydroxyl protecting reagent.

In some embodiments, said providing the compound of formula (III) includes providing a compound of formula (IIB):

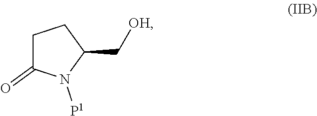

in which P¹ is an N-protecting group and reacting the compound of formula (IIB) with a hydroxyl protecting reagent. In some embodiments, said providing the compound of formula (IIB) comprises reacting (S)-5-(hydroxymethyl)pyrrolidin-2-one with an N-protecting reagent.

In some embodiments, P² in formula (VIII) is a hydroxyl protecting group, e.g., an ester hydroxyl protecting group such as benzoyl, and the method may further include reacting the compound of formula (VIII) with a hydroxyl protecting group removing agent to obtain a compound of formula (IX):

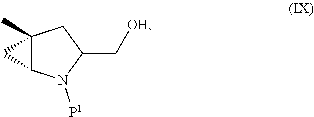

in which P¹ is H or an N-protecting group.

In some embodiments, P¹ in formula (IX) is an N-protecting group, and the method may further include forming a compound of formula (I) from the compound of formula (IX), wherein the compound of formula (I) is of structure:

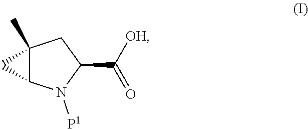

in which P¹ is H or an N-protecting group, and said forming the compound of formula (I) includes oxidizing the compound of formula (IX), e.g., in the presence of (2,2,6,6-tetramethylpiperidin-1-yl)oxyl, sodium hypochlorite, and sodium chlorite.

In some embodiments, the method further includes reacting the compound of formula (I) with an organic amine to form an organoammonium salt of the compound of formula (I), and reacting the organoammonium salt of the compound of formula (I) with an acid to form the compound of formula (I). In some embodiments, the organic amine is benzylamine, and the organoammonium salt is a benzylammonium salt.

In some embodiments, P¹ in formula (I) is an N-protecting group, and the method further includes coupling the compound of formula (I) to a compound of formula (X):

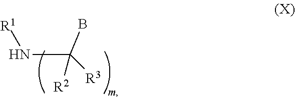

or a salt thereof, in which R¹ is H or optionally substituted C₁-C₆ alkyl; each of R² and R³ is independently H or methyl; m is 0, 1, or 2; and B is optionally substituted C₁-C₆ alkyl, optionally substituted C₂-C₆ alkenyl, optionally substituted C₃-C₁₀ carbocyclyl, optionally substituted C₆-C₁₄ aryl, or optionally substituted 5- to 10-membered heterocyclyl; to form a compound of formula (XI):

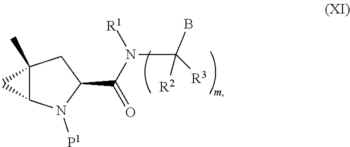

in which P¹ is an N-protecting group and all other variables are as defined for Formula (X), and reacting the compound of formula (XI) with a N-protecting-group-removing agent to form a compound of formula (XII):

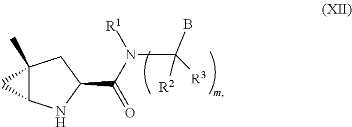

or a salt thereof, wherein all variables are as defined for formula (XI).

In some embodiments, the method further includes coupling the compound of formula (XII) or the salt thereof to a compound of formula (XIII):

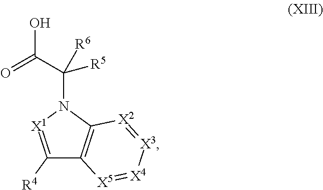

or a salt thereof, in which R⁴ is H; halo; OH; NH₂; cyano; optionally substituted C₁-C₆ alkyl; optionally substituted C₂-C₆ alkenyl; optionally substituted 3- to 8-membered heterocyclyl; —C(O)NRₐRₐ′, wherein each of Rₐ and Rₐ′ is, independently, H, optionally substituted C₁-C₆ alkyl, optionally substituted C₂-C₆ alkenyl, optionally substituted C₂-C₆ alkynyl, or optionally substituted C₃-C₈ cycloalkyl; —C(O)R_{b}; —OC(O)R_{b}; or —C(O)OR_{b}; wherein R_{b}, in each instance, is selected from H, optionally substituted C₁-C₆ alkyl, optionally substituted C₁-C₆ alkoxy, and optionally substituted C₃-C₈ carbocyclyl; each of R⁵ and R⁶ is, independently, H or optionally substituted C₁-C₆ alkyl; X¹ is N or CR_{c}, wherein R_{c} is H, halo, optionally substituted C₁-C₆ alkyl, or optionally substituted C₁-C₆ alkoxy; each of X² and X⁵ is independently N or CR_{d}, wherein each R_{d} is independently selected from H, halo, cyano, optionally substituted C₁-C₆ alkyl, optionally substituted C₁-C₆ alkoxy, optionally substituted C₃-C₈ carbocyclyl, and optionally substituted 5- to 8-membered heteroaryl; and each of X³ and X⁴ is independently selected from N, CRₑ, and CR_{f}, wherein Rₑ is selected from H, halo, cyano, optionally substituted C₁-C₆ alkyl, optionally substituted C₁-C₆ alkoxy, and —C(O)OR_{g}, wherein R_{g} is H or optionally substituted C₁-C₆ alkyl; and R_{f} is selected from optionally substituted C₄-C₁₀ aryl, optionally substituted 5- to 10-membered heteroaryl containing 1, 2, or 3 heteroatoms selected from N, O, and S, and optionally substituted 4- to 10-membered saturated or unsaturated non-aromatic heterocyclyl containing 1-4 heteroatoms selected from N, O, and S; wherein at least one of X³ and X⁴ is CR_{f}; to form a compound of Formula (XIV):

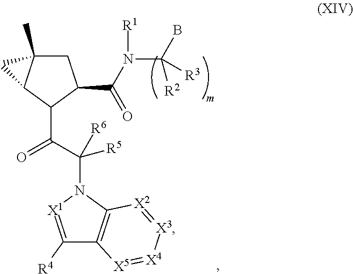

or a pharmaceutically acceptable salt thereof, wherein R¹, R², R³, m, and B are as defined for Formula (XII), and all other variables are as defined for Formula (XIII).

In some embodiments, in which P¹ in each of formula (I) and formula (XI) is tertbutoxycarbonyl.

In some embodiments, in which P¹ in each of formula (I) and formula (XI) is tert-butoxycarbonyl, the N-protecting-group-removing agent may be hydrogen chloride, and said reacting the compound of formula (XI) with the N-protecting-group-removing agent forms a hydrochloride salt of the compound of formula (XII). In some embodiments, the hydrochloride salt of the compound of formula (II) is then coupled to the compound of formula (XIII) in dimethylformamide in the presence of 1-[bis(dimethylamino)methylene]-1H-1,2,3-triazolo[4,5-b]pyridinium 3-oxide hexafluorophosphate and N,N-diisopropylethylamine.

In some embodiments, in which P¹ in each of formula (I) and formula (XI) is tert-butoxycarbonyl, the N-protecting-group-removing agent is hydrogen bromide, and said reacting the compound of formula (XI) with the N-protecting-group-removing agent forms a hydrobromide salt of the compound of formula (XII). In some embodiments, the hydrobromide salt of the compound of formula (XII) is then coupled to the compound of formula (XIII) in acetonitrile in the presence of propanephosphonic acid anhydride and N,N-diisopropylethylamine.

In some embodiments, in which P¹ in each of formula (I) and formula (XI) is tert-butoxycarbonyl, the N-protecting-group-removing agent is trifluoroacetic acid, and said reacting the compound of formula (XI) with the N-protecting-group-removing agent forms a trifluoroacetic acid salt of the compound of formula (XII). In some embodiments, the trifluoroacetic acid salt of the compound of formula (I) is then coupled to the compound of formula (XIII) in dimethylformamide in the presence of N, N-diisopropylethylamine and 1-[bis(dimethylamino)methylene]-1H-1,2,3-triazolo[4,5-b]pyridinium 3-oxide hexafluorophosphate or 2-(1H-benzotriazole-1-yl)-1,1,3,3-tetramethylaminium tetrafluoroborate.

In some embodiments, R¹ is H.

In some embodiments, R¹ is CH₃.

In some embodiments, m is 1.

In some embodiments, m is 2.

In some embodiments, m is 0.

In some embodiments, each of R² and R³ is H.

In some embodiments, R² is H and R³ is CH₃.

In some embodiments, each of R² and R³ is CH₃.

In some embodiments, B is optionally substituted 5- to 10-membered heteroaryl.

In some embodiments, B is optionally substituted 6-membered heteroaryl, e.g., optionally substituted pyridyl, optionally substituted pyridazinyl, optionally substituted pyrimidinyl, or optionally substituted pyrazinyl.

In some embodiments, B is optionally substituted pyridyl, e.g.,

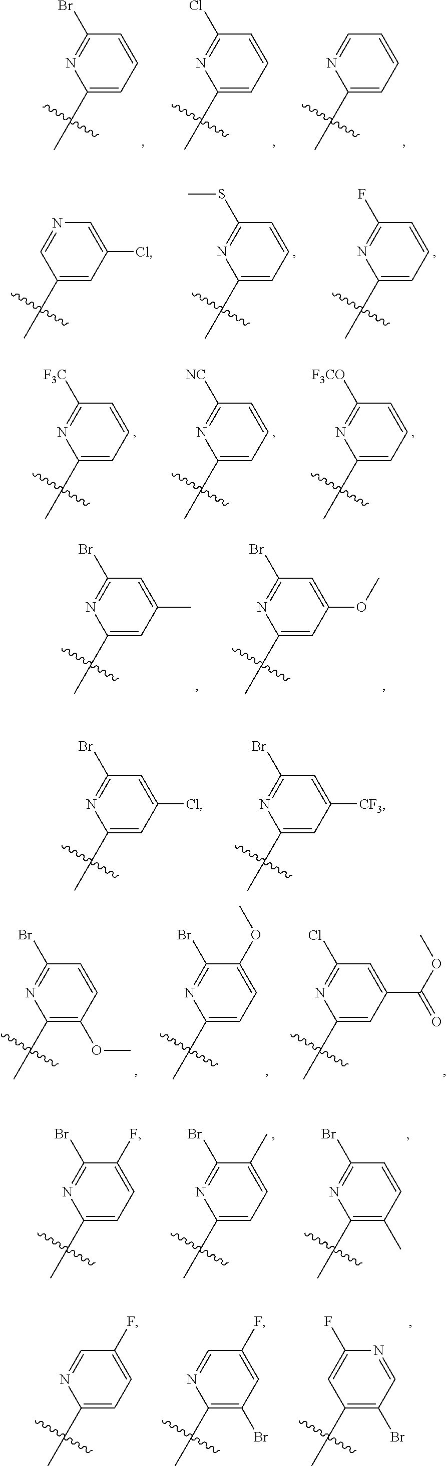

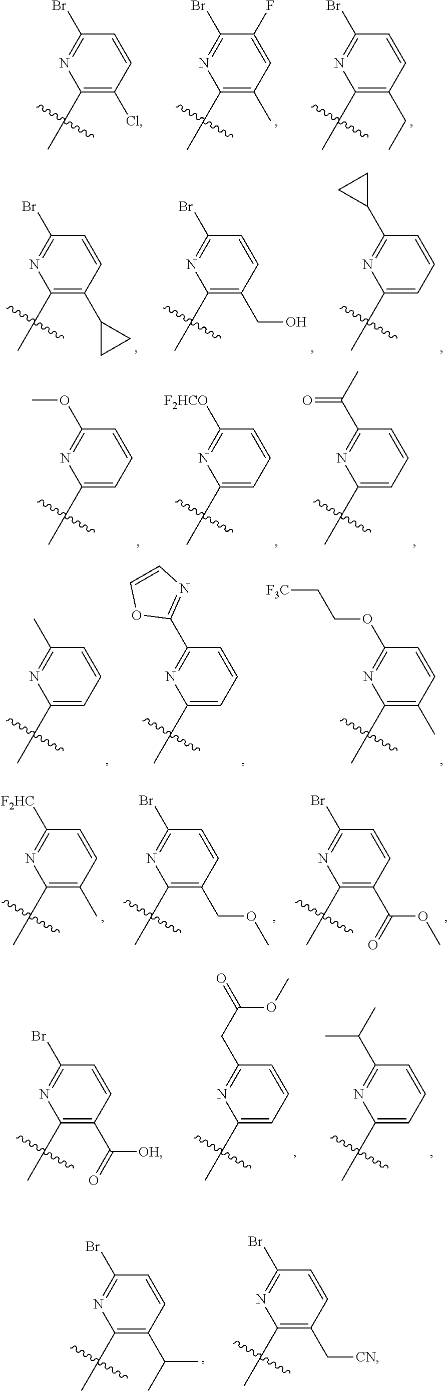

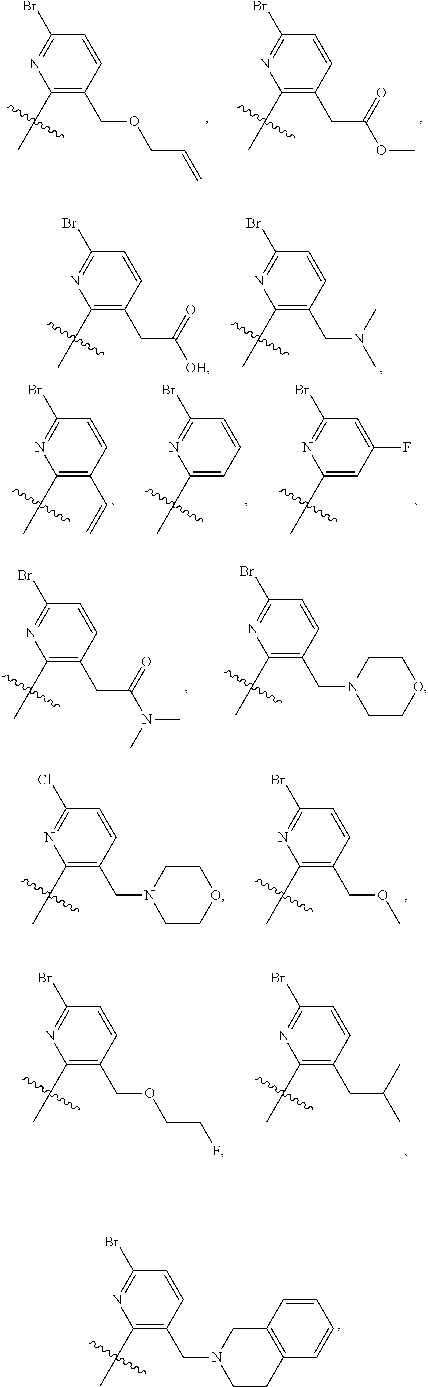

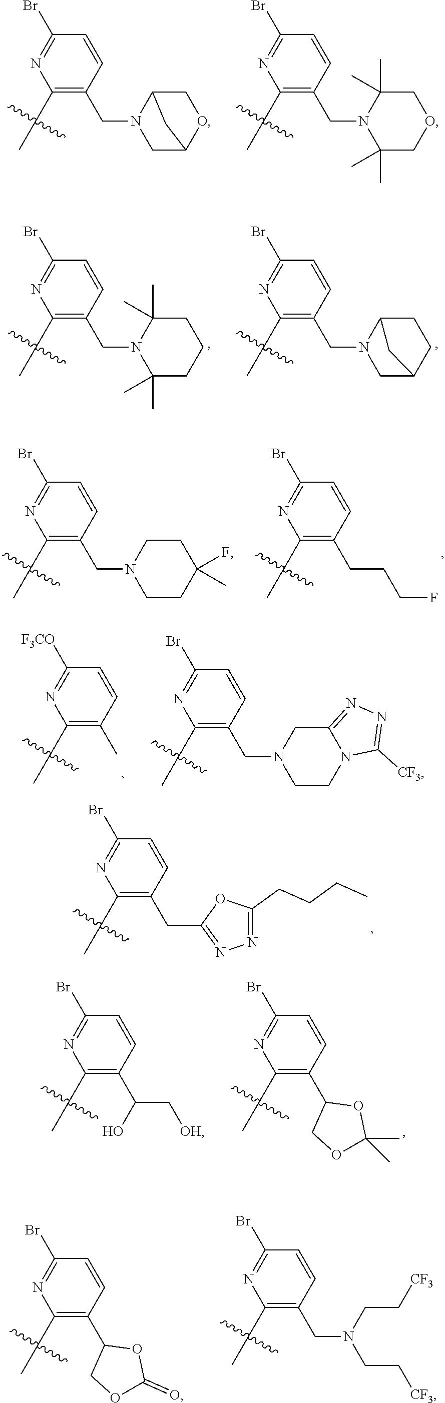

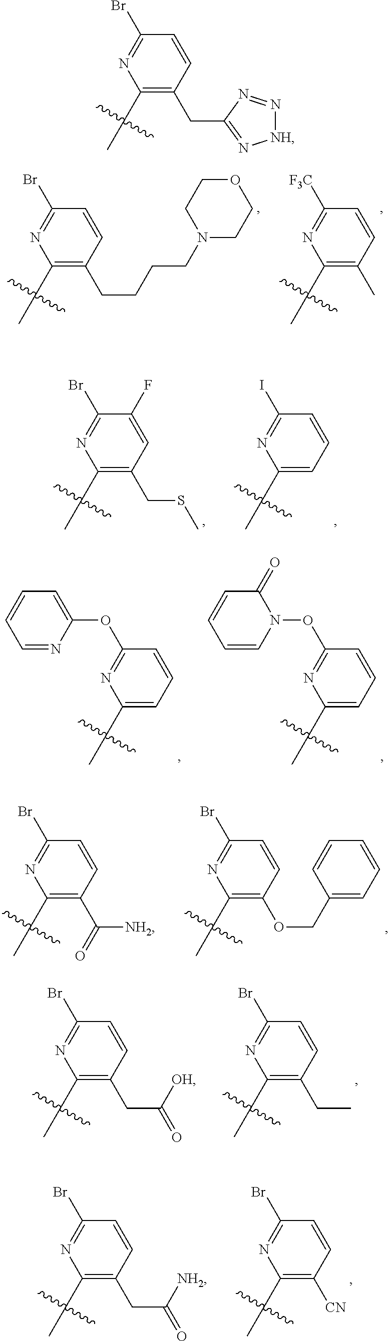

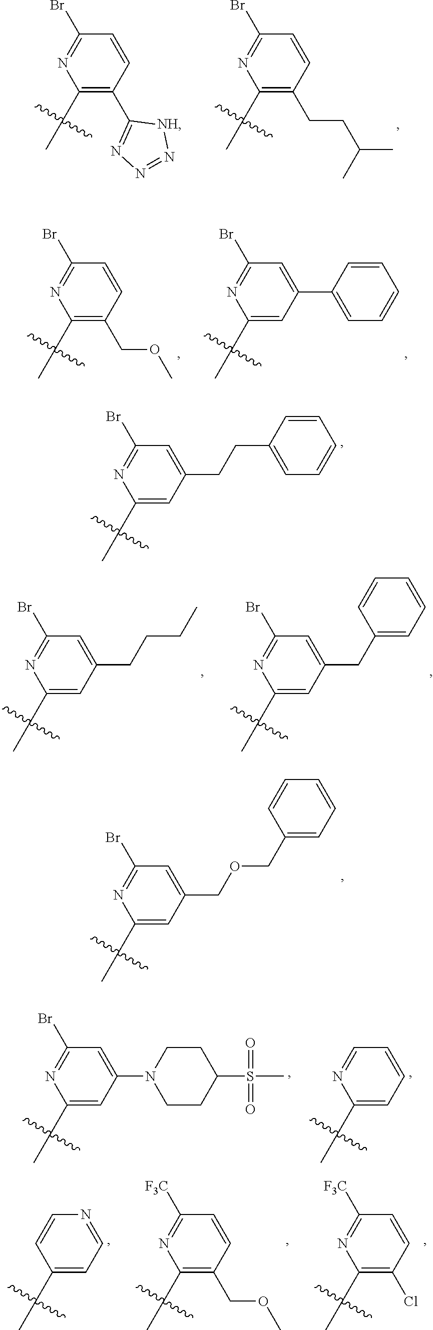

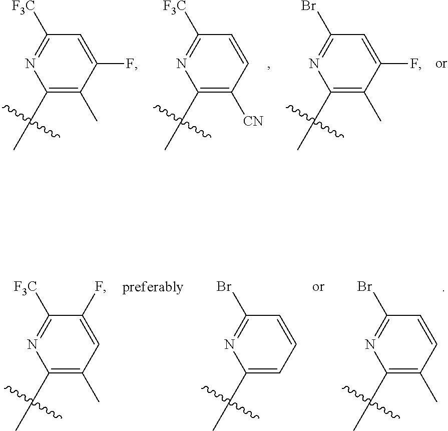

In some embodiments, B is optionally substituted pyrazinyl, e.

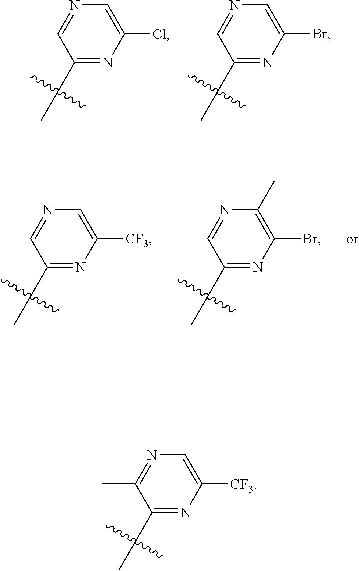

In some embodiments, B is optionally substituted pyrimidinyl, e.g.,

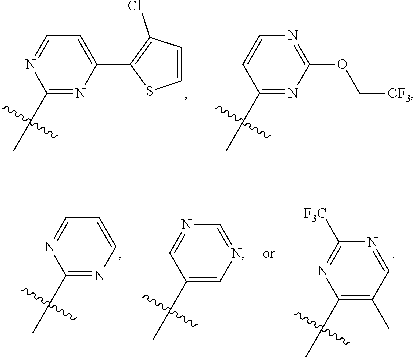 [SMILES: *c1nccc(-c2sccc2Cl)n1 ; *c1ccnc(OCC(F)(F)F)n1 ; *c1ncccn1 ; *c1cncnc1 ; *c1nc(C(F)(F)F)ncc1C]

In some embodiments, B is optionally substituted pyridazinyl, e.g.,

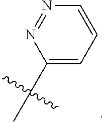 [SMILES: *c1cccnn1]

In some embodiments, B is optionally substituted five-membered heteroaryl, e.g.,

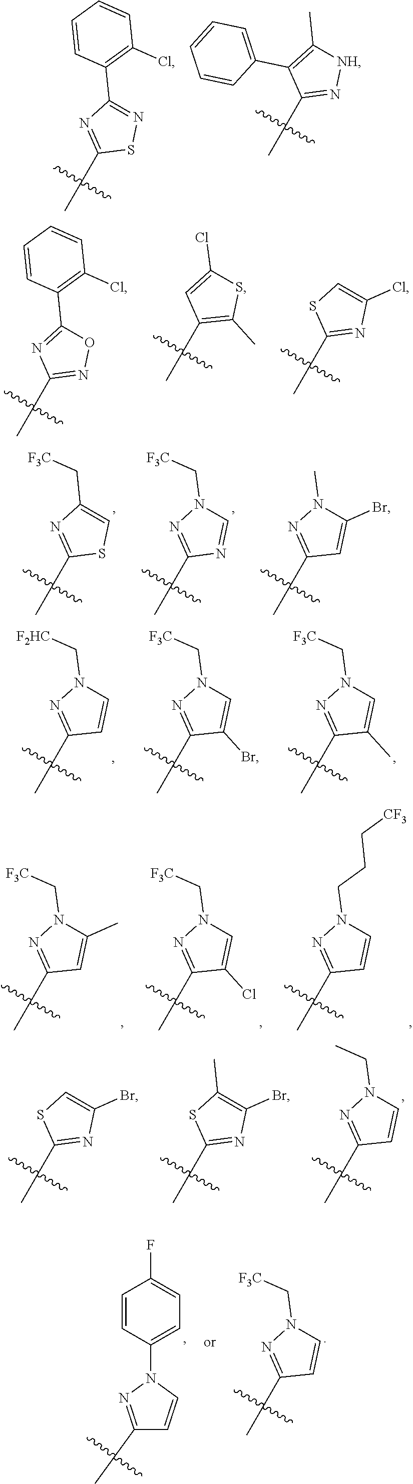

In some embodiments, B is bicyclic 9- or 10-membered bicyclic heteroaryl, e.g.,

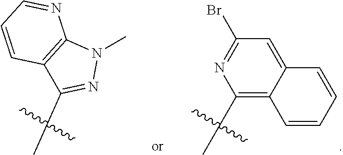 [SMILES: *c1nn(C)c2ncccc12 ; *c1nc(Br)cc2ccccc12]

In some embodiments, B is optionally substituted C₆-C₁₄ aryl, e.g., optionally substituted phenyl, such as

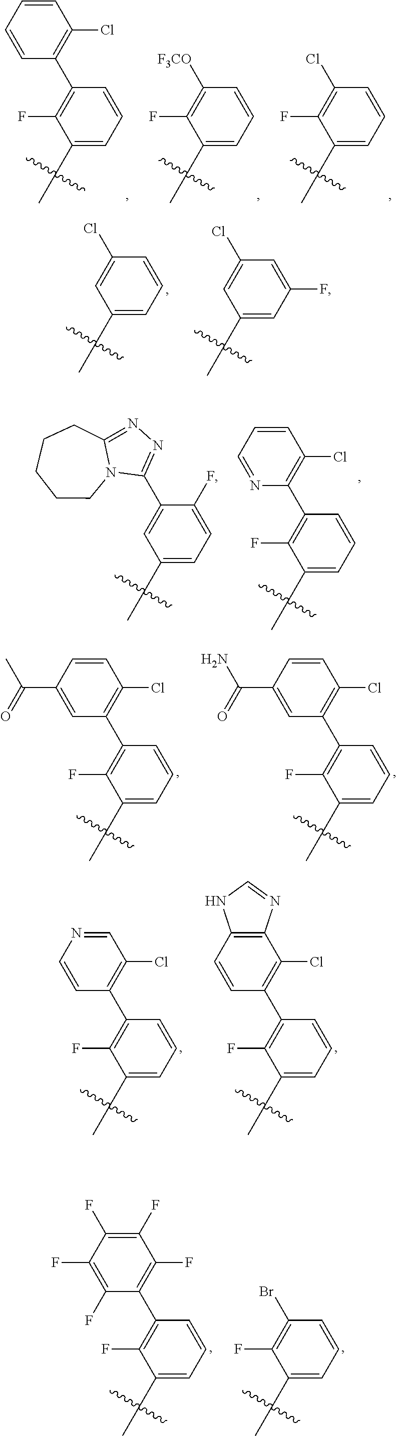

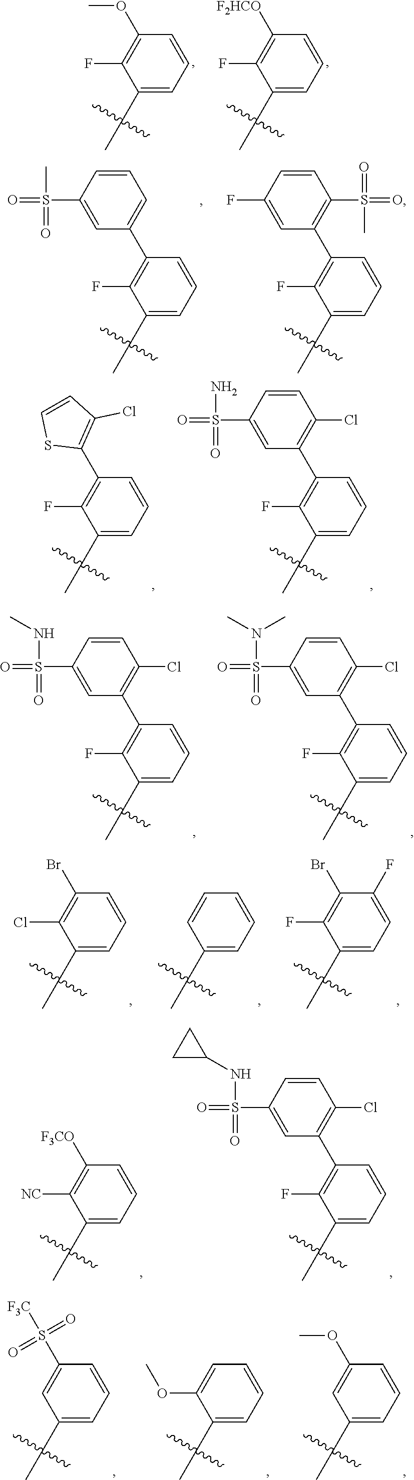

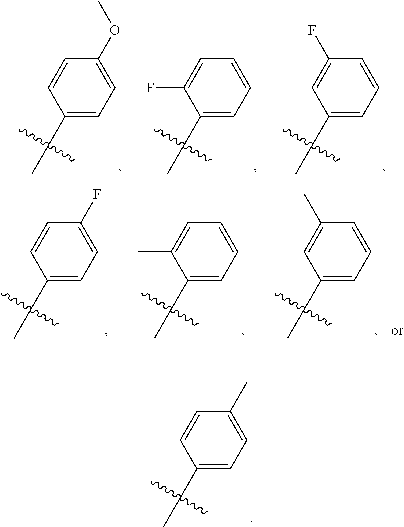

In some embodiments, B is optionally substituted 5- to 9-membered unsaturated heterocyclyl,

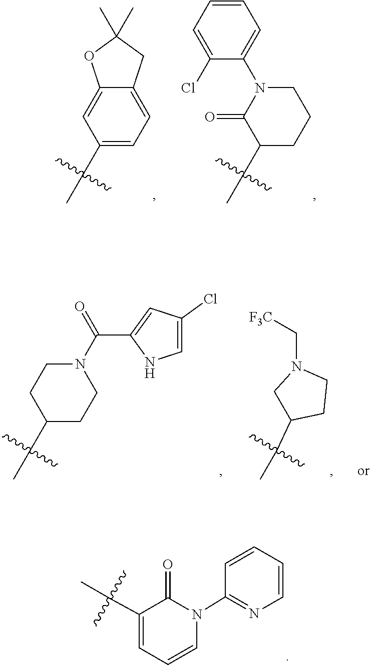 [SMILES: *c1ccc2c(c1)OC(C)(C)C2 ; *C1CCCN(c2ccccc2Cl)C1=O ; *C1CCN(C(=O)c2cc(Cl)c[nH]2)CC1 ; *C1CCN(CC(F)(F)F)C1 ; *c1cccn(-c2ccccn2)c1=O]

In some embodiments, B is optionally substituted C₃-C₁₀ cycloalkyl, e.g.,

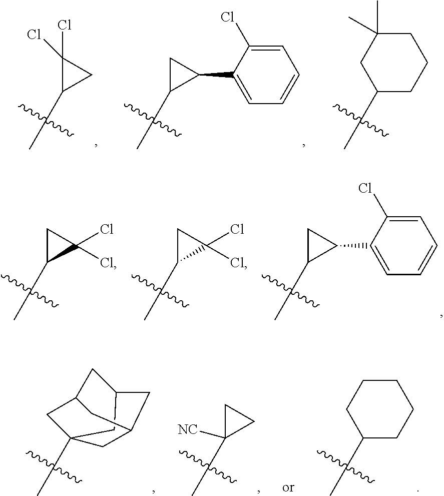

In some embodiments, B is optionally substituted C₂-C₆ alkenyl, e.g.,

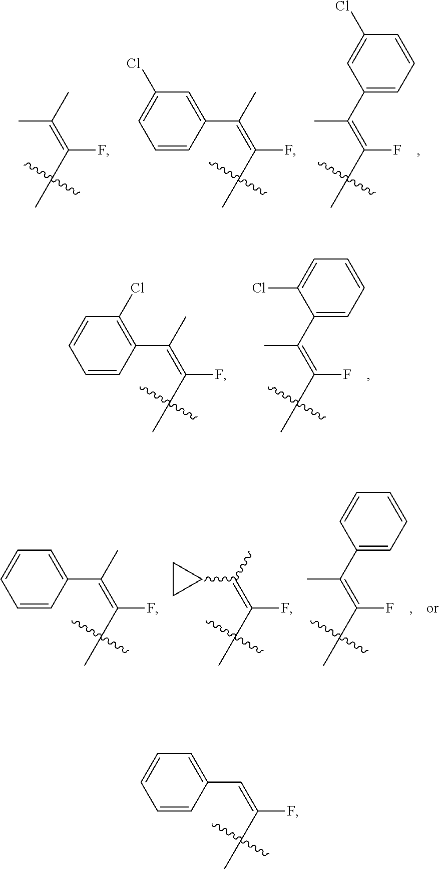

In some embodiments, B is optionally substituted C₁-C₆ alkyl, e.g.,

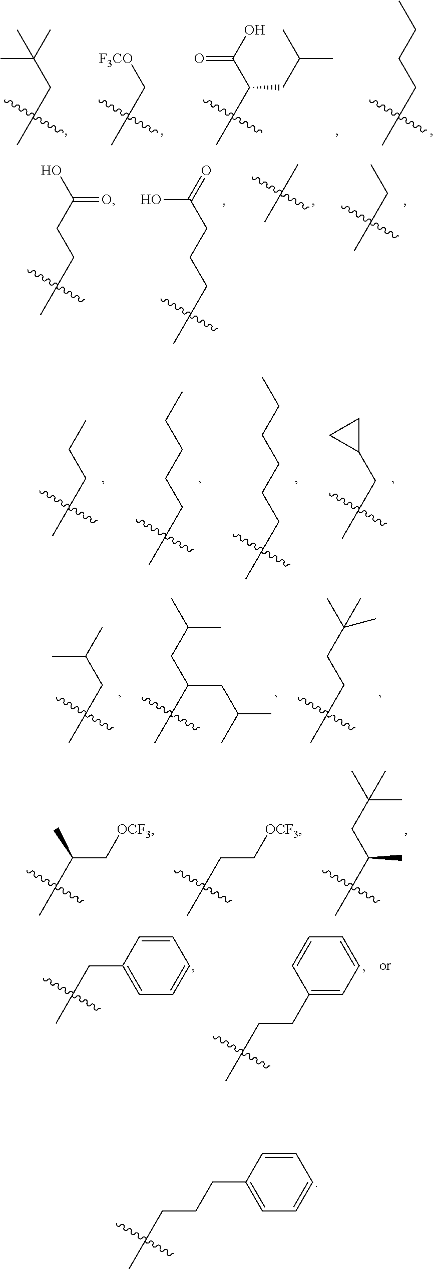

In some embodiments, X¹ is N.

In some embodiments, X¹ is CR_{c}, such as C(CH₃) or CH.

In some embodiments, X² is CR_{d}, such as CH or C(C₁-C₆ alkyl) (e.g., methyl).

In some embodiments, X⁵ is CR_{d}, e.g., CH.

In some embodiments, X³ is CR_{f} and, and X⁴ may be, e.g., N or CH.

In some embodiments, X⁴ is CR_{f} and, and X³ may be, e.g., N or CH.

In some embodiments, R_{f} is optionally substituted 5- to 10-membered heteroaryl containing 1, 2, or 3 heteroatoms selected from N, O, and S.

In some embodiments, R_{f} is 6-membered heteroaryl containing 1, 2, or 3 heteroatoms selected from N, O, and S.

In some embodiments, R_{f} is optionally substituted pyrimidinyl, e.g.,

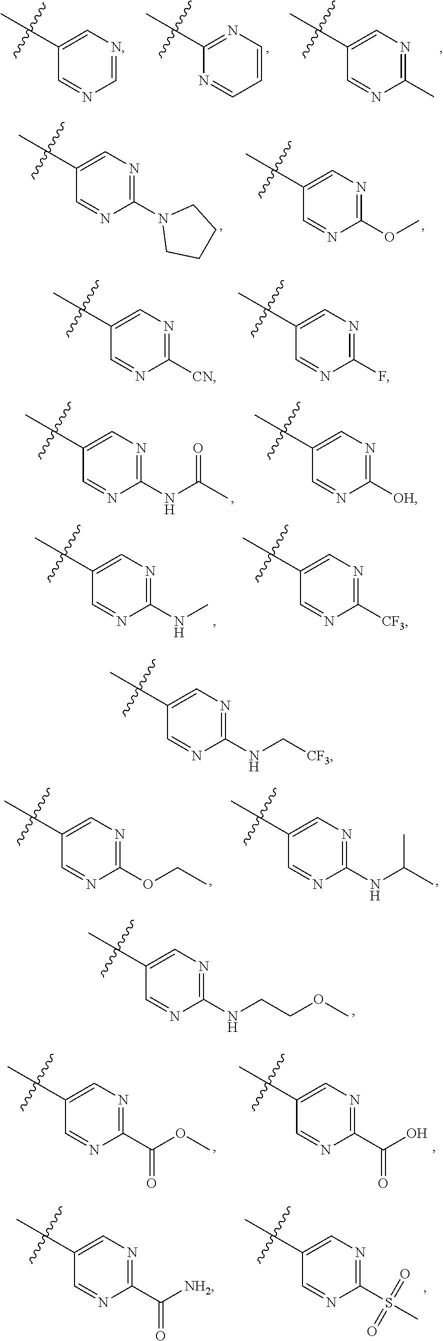

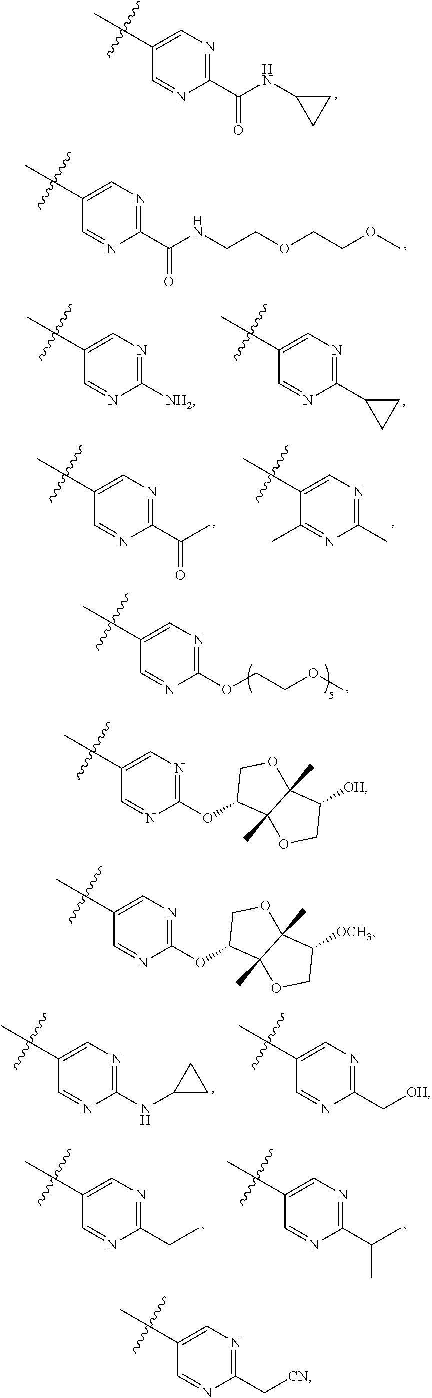

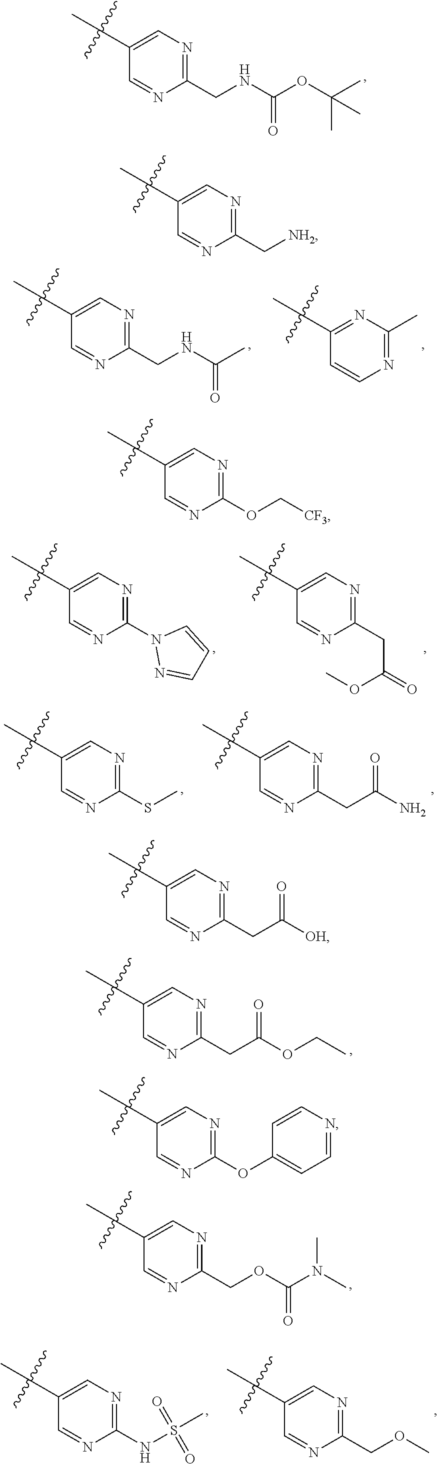

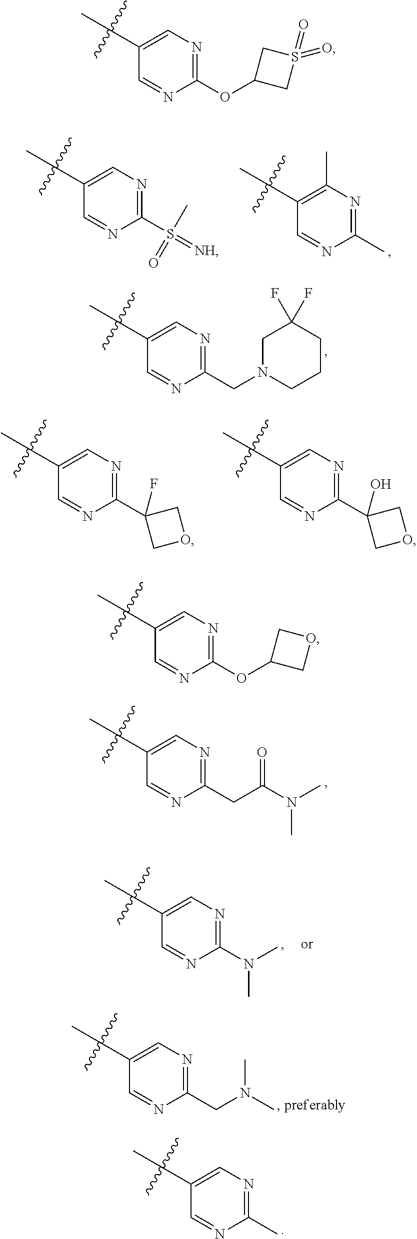

In some embodiments, R_{f} is:

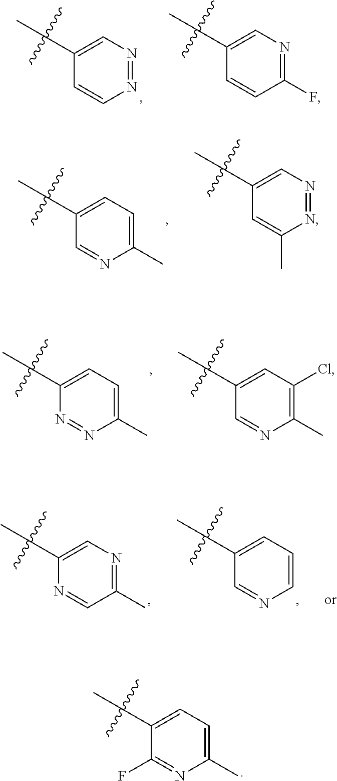

In some embodiments, R_{f} is optionally substituted 8- to 10-membered bicyclic heteroaryl containing 1, 2, or 3 heteroatoms selected from N, O, and S. In some embodiments, R_{f} is optionally substituted pyrazolo[1,5-a]pyrimidinyl, optionally substituted [1,2,4]triazolo[1,5-a]pyridinyl, optionally substituted thiazolo[5,4-b]pyridinyl, optionally substituted imidazo[1,2-a]pyrimidinyl, optionally substituted 3H-imidazo[4,5-b]pyridinyl, 1H-thieno[3,2-c]pyrazolyl, imidazo[1,2-b]pyridazinyl, optionally substituted quinazolinyl, optionally substituted quinolinyl, and 1H-benzo[d]imidazolyl, e.g.,

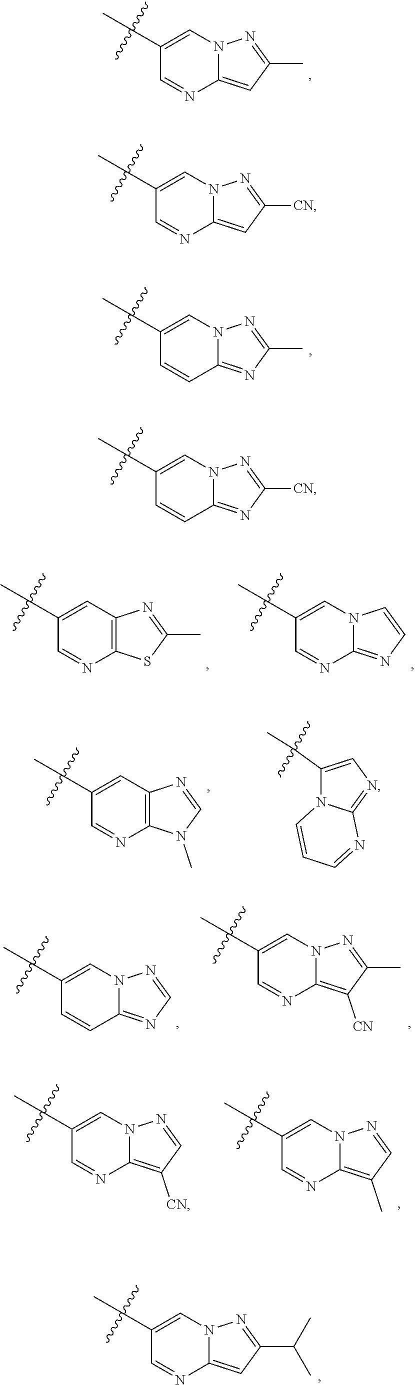

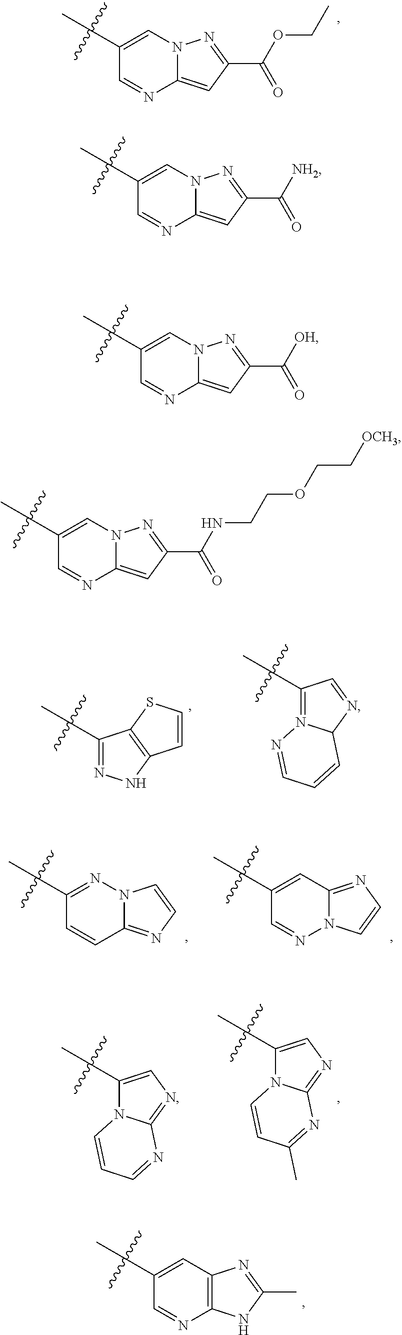

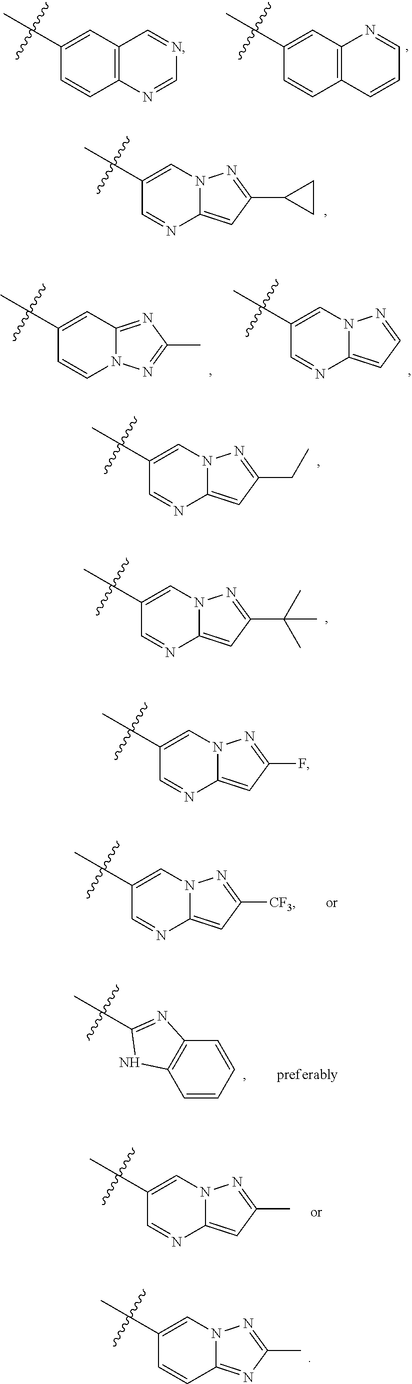

In some embodiments, R_{f} is optionally substituted C₆-C₁₄ aryl, such as optionally substituted phenyl, e.g.,

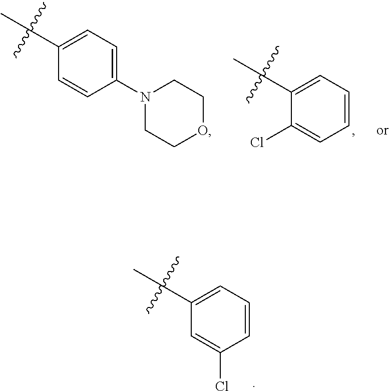 [SMILES: *c1ccc(N2CCOCC2)cc1 ; *c1ccccc1Cl ; *c1cccc(Cl)c1]

In some embodiments, R_{f} is optionally substituted 6- to 9-membered unsaturated heterocyclyl containing 1-4 heteroatoms selected from N, O, or S. For example, R_{f} may be heterocyclyl bonded to the carbon atom to which it is attached through a carbon ring atom contained therein, e.g.,

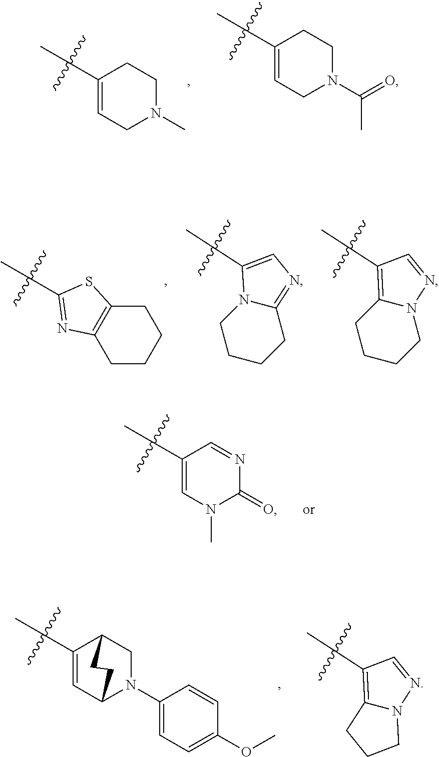

In other examples, R_{f} may be heterocyclyl bonded to the carbon atom to which it is attached through a nitrogen atom contained therein e.g.,

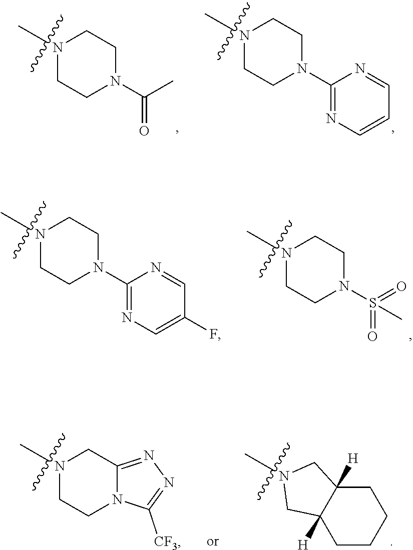

In some embodiments, R_{f} is optionally substituted 5-membered heteroaryl containing 1, 2, or 3 heteroatoms selected from N, O, and S, e.g.,

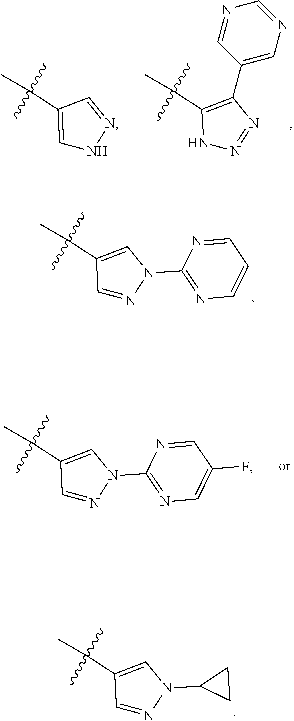 [SMILES: *c1cn[nH]c1 ; *c1[nH]nnc1-c1cncnc1 ; *c1cnn(-c2ncccn2)c1 ; *c1cnn(-c2ncc(F)cn2)c1 ; *c1cnn(C2CC2)c1]

In some embodiments, R⁴ is —C(O)R_{b}, e.g.,

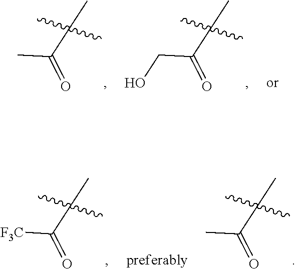

In some embodiments, R⁴ is —C(O)NRₐRₐ′, e.g.,

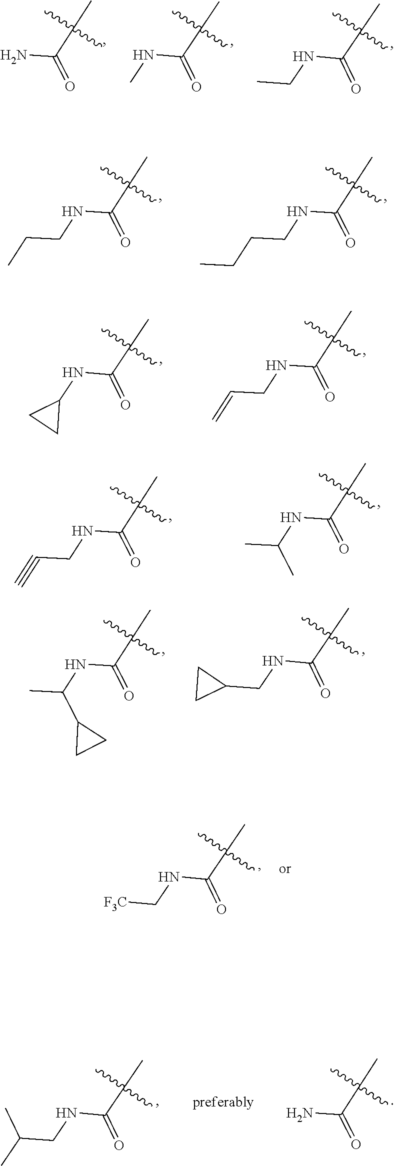

In some embodiments, R⁴ is —C(O)OR_{b}, e.g., —C(O)OCH₃ or —C(O)OH.

In some embodiments, R⁴ is optionally substituted C₁-C₆ alkyl, e.g.,

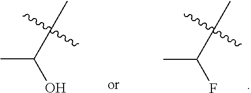

In some embodiments, R⁴ is

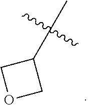 [SMILES: *C1COC1]

In some embodiments, R⁴ is cyano.

In some embodiments, R⁴ is halo.

In some embodiments, R⁴ is H.

In some embodiments, R⁵ is H.

In some embodiments, the compound of formula (XIV) is:

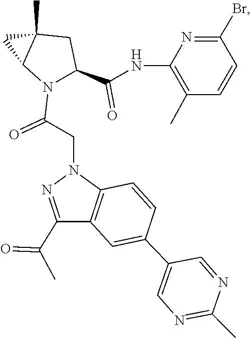 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]

or a pharmaceutically acceptable salt thereof.

In some embodiments, the compound of formula (XIV) is:

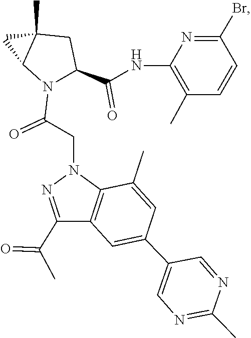 [SMILES: CC(=O)c1nn(CC(=O)N2[C@H](C(=O)Nc3nc(Br)ccc3C)C[C@@]3(C)C[C@@H]23)c2c(C)cc(-c3cnc(C)nc3)cc12]

or a pharmaceutically acceptable salt thereof.

In some embodiments, the compound of formula (XIV) is:

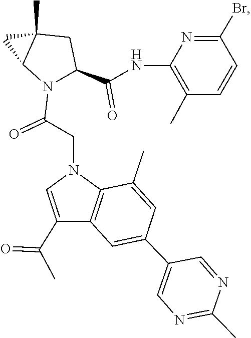 [SMILES: CC(=O)c1cn(CC(=O)N2[C@H](C(=O)Nc3nc(Br)ccc3C)C[C@@]3(C)C[C@@H]23)c2c(C)cc(-c3cnc(C)nc3)cc12]

or a pharmaceutically acceptable salt thereof.

In some embodiments, the compound of formula (XIV) is:

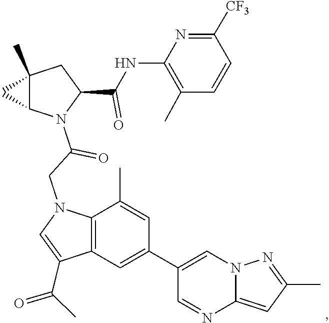 [SMILES: CC(=O)c1cn(CC(=O)N2[C@H](C(=O)Nc3nc(C(F)(F)F)ccc3C)C[C@@]3(C)C[C@@H]23)c2c(C)cc(-c3cnc4cc(C)nn4c3)cc12]

or a pharmaceutically acceptable salt thereof.

In some embodiments, the compound of formula (XIV) is:

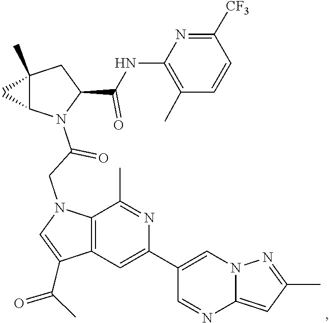 [SMILES: CC(=O)c1cn(CC(=O)N2[C@H](C(=O)Nc3nc(C(F)(F)F)ccc3C)C[C@@]3(C)C[C@@H]23)c2c(C)nc(-c3cnc4cc(C)nn4c3)cc12]

or a pharmaceutically acceptable salt thereof.

In some embodiments, the compound of formula (XIV) is:

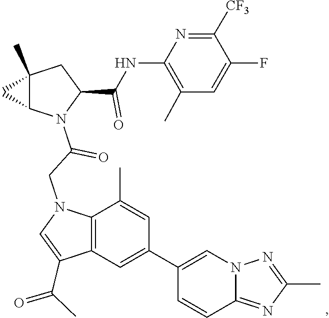 [SMILES: CC(=O)c1cn(CC(=O)N2[C@H](C(=O)Nc3nc(C(F)(F)F)c(F)cc3C)C[C@@]3(C)C[C@@H]23)c2c(C)cc(-c3ccc4nc(C)nn4c3)cc12]

or a pharmaceutically acceptable salt thereof.

In some embodiments, the compound of formula (XIV) is:

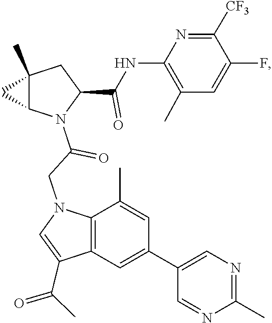 [SMILES: CC(=O)c1cn(CC(=O)N2[C@H](C(=O)Nc3nc(C(F)(F)F)c(F)cc3C)C[C@@]3(C)C[C@@H]23)c2c(C)cc(-c3cnc(C)nc3)cc12]

or a pharmaceutically acceptable salt thereof.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “pharmaceutically acceptable salt” represents those salts of the compounds described that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Handbook of Pharmaceutical Salts: Properties, Selection, and Use, (Eds. P. H. Stahl and C. G. Wermuth), Wiley-VCH, 2008. These salts may be acid addition salts involving inorganic or organic acids. The salts can be prepared in situ during the final isolation and purification of the compounds described herein or separately by reacting the free base group with a suitable acid. Methods for preparation of the appropriate salts are well-established in the art. Representative acid addition salts include acetate, adipate, alginate, ascorbate, aspartate, benzenesulfonate, benzoate, bisulfate, borate, bromide, butyrate, camphorate, camphorsulfonate, chloride, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like.

The term “acyl,” as used herein, refers to a monovalent radical having the structure —COR, where R is alkyl, alkenyl, aryl, arylalkyl, heteroaryl, or heteroarylalkyl. Acyl can be optionally substituted as defined for each R group.

The term “alkyl,” as used herein, refers to a branched or straight-chain monovalent saturated aliphatic radical containing only C and H when unsubstituted. The monovalency of an alkyl group does not include the optional substituents on the alkyl group. For example, if an alkyl group is attached to a compound, monovalency of the alkyl group refers to its attachment to the compound and does not include any additional substituents that may be present on the alkyl group. In some embodiments, the alkyl group may contain, e.g., 1-12, 1-10, 1-8, 1-6, 1-4, or 1-2 carbon atoms (e.g., C₁-C₁₂, C₁-C₁₀, C₁-C₈, C₁-C₆, C₁-C₄, or C₁-C₂). Examples include, but are not limited to, methyl, ethyl, isobutyl, sec-butyl, and tert-butyl.

The term “alkylene,” as used herein, refers to a divalent radical obtained by removing a hydrogen atom from a carbon atom of an alkyl group. The divalency of an alkylene group does not include the optional substituents on the alkylene group.

The term “alkenyl,” as used herein, refers to a branched or straight-chain monovalent unsaturated aliphatic radical containing at least one carbon-carbon double bond and no carbon-carbon triple bonds, and only C and H when unsubstituted. Monovalency of an alkenyl group does not include the optional substituents on the alkenyl group. For example, if an alkenyl group is attached to a compound, monovalency of the alkenyl group refers to its attachment to the compound and does not include any additional substituents that may be present on the alkenyl group. In some embodiments, the alkenyl group may contain, e.g., 2-12, 2-10, 2-8, 2-6, or 2-4 carbon atoms (e.g., C₂-C₁₂, C₂-C₁₀, C₂-C₈, C₂-C₆, or C₂-C₄). Examples include, but are not limited to, ethenyl, 1-propenyl, 2-propenyl, 1-methylethenyl, 1-butenyl, 2-butenyl, 3-butenyl, and the like.

The term “alkenyloxy,” as used herein, refers to a monovalent radical having the structure —O— alkenyl, in which “alkenyl” is as defined herein. Examples include, but are not limited to ethenyloxy, propenyloxy, and the like.

The term “alkoxy,” as used herein, refers to a monovalent radical having the structure —O— alkyl, in which “alkyl” is as defined herein. Examples include, but are not limited to methoxy, ethoxy, and n-butoxy, i-butoxy, t-butoxy, and the like.

The term “alkoxyalkyl,” as used herein, refers to a monovalent radical having the structure —R′OR″, in which R′ is alkylene, and R″ is alkyl. Alkoxyalkyl can be optionally substituted in the same manner as defined for each R′ and R″ group.

The term “alkynyl,” as used herein, refers to a branched or straight-chain monovalent unsaturated aliphatic radical containing at least one carbon-carbon triple bond and only C and H when unsubstituted. Monovalency of an alkynyl group does not include the optional substituents on the alkynyl group. For example, if an alkynyl group is attached to a compound, monovalency of the alkynyl group refers to its attachment to the compound and does not include any additional substituents that may be present on the alkynyl group. In some embodiments, the alkynyl group may contain, e.g., 2-12, 2-10, 2-8, 2-6, or 2-4 carbon atoms (e.g., C₂-C₁₂, C₂-C₁₀, C₂-C₈, C₂-C₆, or C₂-C₄). Examples include, but are not limited to, ethynyl, 1-propynyl, and 3-butynyl.

The term “alkylthioalkyl,” as used herein, refers to a monovalent radical having the structure —R′SR″, in which R′ is alkylene, and R″ is alkyl. Alkylthioalkyl can be optionally substituted in the same manner as defined for each R′ and R″ group.

The term “aryl,” as used herein, refers to a monovalent, monocyclic or fused ring bicyclic or polycyclic system which has the characteristics of aromaticity in terms of electron distribution throughout the ring system, e.g., phenyl, naphthyl, or phenanthryl. An aryl group may have, e.g., six to sixteen carbons (e.g., C₆-C₁₆ aryl, C₆-C₁₄ aryl, C₆-C₁₃ aryl, or C₆-C₁₀ aryl).

The term “arylalkoxy,” as used herein, refers to a monovalent radical having the structure—OR′R″, where R′ is alkylene, and R″ is aryl. Arylalkoxy can be optionally substituted in the same manner as defined for each R′ and R″ group.

The term “arylalkoxyalkyl,” as used herein, refers to a monovalent radical having the structure —R′OR′R″, where each R′ is alkylene, and R″ is aryl. Arylalkoxyalkyl can be optionally substituted in the same manner as defined for each R′ and R″ group.

The term “arylalkyl,” as used herein, refers to a monovalent radical having the structure —R′R″, where R′ is alkylene, and R″ is aryl. Arylalkyl can be optionally substituted in the same manner as defined for each R′ and R″ group.

The term “arylene,” as used herein, refers to a divalent radical obtained by removing a hydrogen atom from a carbon atom of an aryl group. The divalency of an arylene group does not include the optional substituents on the arylene group.

The term “carbamate,” as used herein, represents a monovalent radical having the structure formula —OC(O)NR₂, in each R is independently H, optionally substituted alkyl, optionally substituted alkenyl, optionally substituted aryl, or optionally substituted arylalkyl.

The term “carbocyclyl,” as used herein, represents a monovalent, saturated or unsaturated non-aromatic cyclic group containing only C and H when unsubstituted. A carbocyclyl (e.g., a cycloalkyl or a cycloalkenyl) may have, e.g., three to fourteen carbons (e.g., a C₃-C₇, C₃-C₈, C₃-C₉, C₃-C₁₀, C₃-C₁₁, C₃-C₁₂, C₃-C₁₄ carbocyclyl). The term “carbocyclyl” also includes bicyclic and polycyclic (e.g., tricyclic and tetracyclic) fused ring structures.

The term “carbocyclyene,” as used herein, refers to a divalent radical obtained by removing a hydrogen atom from a carbon atom of a carbocyclyl group. The divalency of a carbocyclylene group does not include the optional substituents on the carbocyclylene group

The term “carbocyclyloxy,” as used herein, refers to a monovalent radical having the structure —O-carbocyclyl, e.g., a —O-cycloalkyl or a —O-cycloalkenyl radical. The terms “carbocyclyl,” “cycloalkyl,” and “cycloalkenyl” included in —O-carbocyclyl, —O-cycloalkyl, and —O-cycloalkenyl are as defined herein.

The term “carbonate,” as used herein, whrefers to a monovalent radical having the structure —OC(O)OR, where R is optionally substituted alkyl, optionally substituted alkenyl, optionally substituted aryl, or optionally substituted arylalkyl.

The term “cycloalkyl”, as used herein refers to a saturated carbocyclyl. Examples of cycloalkyls include, but are not limited to, cyclopropyl, cyclobutyl, cyclopentyl, cyclohexyl, and cycloheptyl. The term “cycloalkyl” also includes cyclic groups having a bridged multicyclic structure in which one or more carbons bridges two non-adjacent members of a monocyclic ring, e.g., bicyclo[2.2.1]heptyl and adamantyl. The term “cycloalkyl” also includes bicyclic, tricyclic, and tetracyclic fused ring structures, e.g., decalin and spirocyclic compounds.

The term “cyano,” as used herein, refers to a monovalent radical having the structure —CN.

The term “cycloalkenyl,” as used herein, represents a monovalent, unsaturated carbocyclyl group that includes at least one carbon-carbon double bond, no carbon-carbon triple bond, only C and H when unsubstituted, and is not fully aromatic. A cycloalkenyl may have, e.g., four to fourteen carbons (e.g., a C₄-C₇, C₄-C₈, C₄-C₉, C₄-C₁₀, C₄-C₁₁, C₄-C₁₂, C₄-C₁₃, or C₄-C₁₄ cycloalkenyl). Exemplary cycloalkenyl groups include, but are not limited to, cyclopentenyl, cyclohexenyl, and cycloheptenyl. The term “cycloalkenyl” also includes cyclic groups having a bridged multicyclic structure in which one or more carbons bridges two non-adjacent members of a monocyclic ring, e.g., bicyclo[2.2.2]oct-2-ene. The term “cycloalkenyl” also includes fused ring bicyclic and multicyclic systems containing one or more double bonds, e.g., fluorene.

The term “ester,” as used herein, refers to a monovalent radical having the structure —OCOR, where R is alkyl, alkenyl, aryl, arylalkyl, heteroaryl, or heteroarylalkyl. Ester can be optionally substituted as defined for each R group.

The term “ether,” as used herein, refers to a monovalent radical having the structure —OR, where R is alkyl, alkenyl, arylalkyl, silyl, or 2-tetrahydropyranyl. Ether can be optionally substituted as defined for each R group.

The term “halo,” as used herein, refers to a fluorine (fluoro), chlorine (chloro), bromine (bromo), or iodine (iodo) radical.

The term “heteroarylalkyl,” as used herein, represents a monovalent radical of structure —R′R″, where R′ is alkylene, and R″ is heteroaryl. Heteroarylalkyl can be optionally substituted in the same manner as defined for each R′ and R″ group.

The term “heterocyclyl,” as used herein, represents a saturated or unsaturated monocyclic or fused ring bicyclic or polycyclic system having one or more carbon atoms and at least one heteroatom, e.g., one to four heteroatoms (e.g., one to four, one to three, one or two, one, two, three, or four heteroatoms), selected from N, O, and S. Heterocyclyl groups include both non-aromatic and aromatic systems. An aromatic heterocyclyl group is referred to as a “heteroaryl” group. In some embodiments, a heterocyclyl group is a 3- to 8-membered ring system, a 3- to 6-membered ring system, a 4- to 6-membered ring system, a 4- to 10-membered ring system, a 6- to 10-membered ring system, a 6- to 12-membered ring system, a 5-membered ring, or a 6-membered ring, or a ring or ring system having a number of ring atoms that fall within any of the above-mentioned ranges. Exemplary 5-membered heterocyclyl groups may have zero to two double bonds, and exemplary 6-membered heterocyclyl groups may have zero to three double bonds. Exemplary 5-membered groups include, for example, optionally substituted pyrrole, optionally substituted pyrazole, optionally substituted isoxazole, optionally substituted pyrrolidine, optionally substituted imidazole, optionally substituted thiazole, optionally substituted thiophene, optionally substituted thiolane, optionally substituted furan, optionally substituted tetrahydrofuran, optionally substituted diazole, optionally substituted triazole, optionally substituted tetrazole, optionally substituted oxazole, optionally substituted 1,3,4-oxadiazole, optionally substituted 1,3,4-thiadiazole, optionally substituted 1,2,3,4-oxatriazole, and optionally substituted 1,2,3,4-thiatriazole. Exemplary 6-membered heterocyclyl groups include, but are not limited to, optionally substituted pyridine, optionally substituted piperidine, optionally substituted piperazine, optionally substituted pyrimidine, optionally substituted pyrazine, optionally substituted pyridazine, optionally substituted triazine, optionally substituted 2H-pyran, optionally substituted 4H-pyran, and optionally substituted tetrahydropyran. Exemplary 7-membered heterocyclyl groups include, but are not limited to, optionally substituted azepine, optionally substituted 1,4-diazepine, optionally substituted thiepine, and optionally substituted 1,4-thiazepine. Exemplary 8- to 10-membered bicyclic groups include, but are not limited to, optionally substituted pyrazolo[1,5-a]pyrimidinyl, optionally substituted [1,2,4]triazolo[1,5-a]pyridinyl, optionally substituted thiazolo[5,4-b]pyridinyl, optionally substituted imidazo[1,2-a]pyrimidinyl, optionally substituted 3H-imidazo[4,5-b]pyridinyl, 1H-thieno[3,2-c]pyrazolyl, imidazo[1,2-b]pyridazinyl, optionally substituted quinazolinyl, optionally substituted quinolinyl, and 1H-benzo[d]imidazolyl.

The term “hydroxyl protecting group,” as used herein, refers to any group capable of protecting the oxygen atom to which it is attached from reacting or bonding. A hydroxyl protecting group is installed by reacting a molecule including an unprotected hydroxyl group with a hydroxyl-protecting reagent. Hydroxyl protecting groups are known in the art, e.g., as described in Wuts, Greene's Protective Groups in Organic Synthesis, Wiley-Interscience, 4th Edition, 2006. Exemplary protecting groups (with the oxygen atom to which they are attached) are independently selected from the group consisting of esters, carbonates, carbamates, sulfonates, and ethers. In exemplary ester hydroxyl protecting groups, R of the acyl group is C₁-C₁₂ alkyl (e.g., C₁-C₈, C₁-C₆, C₁-C₄, C₂-C₇, C₃-C₁₂, and C₃-C₆ alkyl), C₂-C₁₂ alkenyl (e.g., C₂-C₈, C₂-C₆, C₂-C₄, C₃-C₁₂, and C₃-C₆ alkenyl), carbocyclic C₆-C₂₀ aryl (e.g., C₆-C₁₅, C₆-C₁₀, C₃-C₂₀, and C₈-C₁₅ aryl), monocyclic C₁-C₆ heteroaryl (e.g., C₁-C₄ and C₂-C₆ heteroaryl), C₄-C₁₉ heteroaryl (e.g., C₄-C₁₀ heteroaryl), (C₆-C₁₅)aryl(C₁-C₆)alkyl, (C₄-C₁₉)heteroaryl(C₁-C₆)alkyl, or (C₁-C₆)heteroaryl(C₁-C₆)alkyl. Specific examples of acyl groups for use in esters include formyl, benzoylformyl, acetyl (e.g., unsubstituted or chloroacetyl, trifluoroacetyl, methoxyacetyl, triphenylmethoxyacetyl, and p-chlorophenoxyacetyl), 3-phenylpropionyl, 4-oxopentanoyl, 4,4-(ethylenedithio)pentanoyl, pivaloyl (Piv), vinylpivaloyl, crotonoyl, 4-methoxy-crotonoyl, naphthoyl (e.g., 1- or 2-naphthoyl), and benzoyl (e.g., unsubstituted or substituted, e.g., p-methoxybenzoyl, phthaloyl (including salts, such a triethylamine and potassium), p-bromobenzoyl, and 2,4,6-trimethylbenzoyl). As defined herein, any heteroaryl group present in an ester group has from 1 to 4 heteroatoms selected independently from O, N, and S. In exemplary carbonate hydroxyl protecting groups, R is C₁-C₁₂ alkyl (e.g., C₁-C₈, C₁-C₆, C₁-C₄, C₂-C₇, C₃-C₁₂, and C₃-C₆ alkyl), C₂-C₁₂ alkenyl (e.g., C₂-C₈, C₂-C₆, C₂-C₄, C₃-C₁₂, and C₃-C₆ alkenyl), carbocyclic C₈-C₂₀ aryl (e.g., C₆-C₁₅, C₆-C₁₀, C₃-C₂₀, and C₈-C₁₅ aryl), monocyclic C₁-C₆ heteroaryl (e.g., C₁-C₄ and C₂-C₆ heteroaryl), C₄-C₁₉ heteroaryl (e.g., C₄-C₁₀ heteroaryl), (C₆-C₁₅)aryl(C₁-C₆)alkyl, (C₄-C₁₉)heteroaryl(C₁-C₆)alkyl, or (C₁-C₆)heteroaryl(C₁-C₆)alkyl. Specific examples include methyl, 9-fluorenylmethyl, ethyl, 2,2,2-trichloroethyl, 2-(trimethylsilyl)ethyl, 2-(phenylsulfonyl)ethyl, vinyl, allyl, t-butyl, p-nitrobenzyl, and benzyl carbonates. As defined herein, any heteroaryl group present in a carbonate group has from 1 to 4 heteroatoms selected independently from O, N, and S. In exemplary carbamate hydroxyl protecting groups, each R is independently H, C₁-C₁₂ alkyl (e.g., C₁-C₈, C₁-C₆, C₁-C₄, C₂-C₇, C₃-C₁₂, and C₃-C₆ alkyl), C₂-C₁₂ alkenyl (e.g., C₂-C₈, C₂-C₆, C₂-C₄, C₃-C₁₂, and C₃-C₆ alkenyl), carbocyclic C₆-C₂₀ aryl (e.g., C₆-C₁₅, C₆-C₁₀, C₈-C₂₀, and C₈-C₁₅ aryl), monocyclic C₁-C₆ heteroaryl (e.g., C₁-C₄ and C₂-C₆ heteroaryl), C₄-C₁₉ heteroaryl (e.g., C₄-C₁₀ heteroaryl), (C₆-C₁₅)aryl(C₁-C₆)alkyl, (C₄-C₁₉)heteroaryl(C₁-C₆)alkyl, or (C₁-C₆)heteroaryl(C₁-C₆)alkyl. Specific examples include N-phenyl and N-methyl-N-(o-nitrophenyl) carbamates. As defined herein, any heteroaryl group present in a carbamate group has from 1 to 4 heteroatoms selected independently from O, N, and S. Exemplary ether hydroxyl protecting groups include C₁-C₁₂ alkyl (e.g., C₁-C₈, C₁-C₆, C₁-C₄, C₂-C₇, C₃-C₁₂, and C₃-C₆ alkyl), C₂₋₁₂ alkenyl (e.g., C₂-C₈, C₂-C₆, C₂-C₄, C₃-C₁₂, and C₃-C₆ alkenyl), (C₆-C₁₅)aryl(C₁-C₆)alkyl, (C₄-C₁₉)heteroaryl(C₁-C₆)alkyl, (C₁-C₆)heteroaryl(C₁-C₆)alkyl, (C₁-C₆)alkoxy(C₁-C₆)alkyl, (C₁-C₆)alkylthio(C₁-C₆)alkyl, (C₆-C₁₀)aryl(C₁-C₆)alkoxy(C₁-C₆)alkyl, and silyl (e.g., tri(C₁-C₆ alkyl)silyl, tri(C₆-C₁₀ aryl or C₁-C₆ heteroaryl)silyl, di(C₆-C₁₀ aryl or C₁-C₆ heteroaryl)(C₁-C₆ alkyl)silyl, and (C₆-C₁₀ aryl or C₁-C₆ heteroaryl)di(C₁-C₆ alkyl)silyl). Specific examples of alkylethers include methyl and t-butyl, and an example of an alkenyl ether is allyl. Ether hydroxyl protecting groups can be used to protect a carboxyl group (e.g., with a C₁₋₁₂ alkyl (e.g., C₁₋₈, C₁₋₆, C₁₋₄, C₂₋₇, C₃₋₁₂, and C₃₋₆ alkyl), (C₆₋₆) aryl(C₁₋₆)alkyl, (C₁₋₆)alkoxy(C₁₋₆)alkyl, (C₁₋₆)alkylthio(C₁₋₆)alkyl, or (C₆₋₁₀)aryl(C₁₋₆)alkoxy(C₁₋₆)alkyl). Examples of alkoxyalkyls and alkylthioalkyls that can be used as ether hydroxyl protecting groups include methoxymethyl, methylthiomethyl, (2-methoxyethoxy)methyl, and β-(trimethylsilyl)ethoxymethyl. Examples of arylalkyl groups that can be used as ether hydroxyl protecting groups include benzyl, p-methoxybenzyl (MPM), 3,4-dimethoxybenzyl, triphenylmethyl (trityl), o-nitrobenzyl, p-nitrobenzyl, p-halobenzyl, 2,6-dichlorobenzyl, p-cyanobenzyl, naphthylmethyl, and 2- and 4-picolyl ethers. Specific examples of silylethers include trimethylsilyl (TMS), triethylsilyl (TES), t-butyldimethylsilyl (TBS), t-butyldiphenylsilyl (TBDPS), triisopropylsilyl (TIPS), and triphenylsilyl (TPS) ethers. An example of an arylalkyloxyalkylether is benzyloxymethyl ether. As defined herein, any heteroaryl group present in an ether group has from 1 to 4 heteroatoms selected independently from O, N, and S. Alkyl groups, such as methyl, ethyl, isopropyl, n-propyl, t-butyl, n-butyl, and sec-butyl, and alkenyl groups, such as vinyl and allyl, can also be substituted with oxo, arylsulfonyl, halogen, and trialkylsilyl groups. Protecting groups can be installed and removed using methods known in the art.

The term “N-protecting group,” as used herein, refers to a group protecting a nitrogen atom in a molecule from participating in one or more undesirable reactions during chemical synthesis (e.g., oxidation reactions, or certain nucleophilic and electrophilic substitutions). An N-protecting group is installed by reacting the molecule including a nitrogen atom with an N-protecting reagent. Commonly used N-protecting groups and the corresponding N-protecting reagents are disclosed in Wuts, Greene's Protective Groups in Organic Synthesis, Wiley-Interscience, 4th Edition, 2006. Exemplary N-protecting groups include acyl (e.g., formyl, acetyl, trifluoroacetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, and 4-bromobenzoyl); sulfonyl-containing groups (e.g., benzenesulfonyl, p-toluenesulfonyl, o-nitrobenzenesulfonyl, and p-nitrobenzenesulfonyl); carbamate forming groups (e.g., benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl), arylalkyl (e.g., triphenylmethyl); silyl groups (e.g., trimethylsilyl); and imine-forming groups (e.g., diphenylmethylene). Preferred N-protecting groups are acetyl, benzoyl, phenylsulfonyl, p-toluenesulfonyl, p-nitrobenzenesulfonyl, o-nitrobenzenesulfonyl, t-butyloxycarbonyl (Boc), and benzyloxycarbonyl (Cbz).

The term “oxo,” as used herein, refers to a divalent oxygen atom represented by the structure ═O.

The term “silyl,” as used herein refers to a group of —SiR₃, in which each R is independently alkyl, alkenyl, aryl, or arylalkyl. Examples of silyl groups include tri(C₁-C₆ alkyl)silyl, tri(C₆-C₁₀ aryl or C₁-C₆ heteroaryl)silyl, di(C₆₋₁₀ aryl or C₁-C₆ heteroaryl)(C₁-C₆ alkyl)silyl, and (C₆-C₁₀ aryl or C₁-C₆ heteroaryl)di(C₁-C₆ alkyl)silyl. It will be understood that, when a silyl group includes two or more alkyl, alkenyl, aryl, heteroaryl, or arylalkyl groups, these groups are independently selected. As defined herein, any heteroaryl group present in a silyl group has from 1 to 4 heteroatoms selected independently from O, N, and S. Silyl can be optionally substituted in the same manner as defined for each R group.

The term “sulfonyl,” as defined herein, refers to a group of —S(O)₂R, where R is optionally substituted alkyl, optionally substituted alkenyl, optionally substituted aryl, optionally substituted arylalkyl, or silyl. In exemplary sulfonyls, R is C₁-C₁₂ alkyl (e.g., C₁-C₈, C₁-C₆, C₁-C₄, C₂-C₇, C₃-C₁₂, or C₃-C₆ alkyl), C₂-C₁₂ alkenyl (e.g., C₂-C₈, C₂-C₆, C₂-C₄, C₃-C₁₂, or C₃-C₆ alkenyl), carbocyclic C₆-C₂₀ aryl (e.g., C₆-C₁₅, C₆-C₁₀, C₈-C₂₀, or C₈-C₁₅ aryl), monocyclic C₁-C₆ heteroaryl (e.g., C₁-C₄ and C₂-C₆ heteroaryl), C₄-C₁₉ heteroaryl (e.g., C₄-C₁₀ heteroaryl), (C₆-C₁₅)aryl(C₁-C₆)alkyl, (C₄-C₁₉)heteroaryl(C₁-C₆)alkyl, or (C₁-C₆)heteroaryl(C₁-C₆)alkyl. As defined herein, any heteroaryl group present in a sulfonate group has from 1 to 4 heteroatoms selected independently from 0, N, and S.

The term “thioalkyl,” as used herein, refers to a monovalent radical having the structure —S— alkyl, in which “alkyl” is as defined herein.

The phrase “optionally substituted X,” as used herein, is intended to be equivalent to “X, wherein X is optionally substituted” (e.g., “alkyl, wherein said alkyl is optionally substituted”). It is not intended to mean that the feature “X” (e.g. alkyl) per se is optional. The term “optionally substituted,” as used herein, refers to having 0, 1, or more substituents (e.g., 0-10, 0-9, 0-8, 0-7, 0-6, 0-5, 0-4, 0-3, 0-2, 0 or 1, 0, 1, 2, 3, 4, 5, 6, 7, 8, 9, or 10 substituents).

Alkyl, alkylene, alkenyl, alkynyl, carbocyclyl, cycloalkyl, cycloalkenyl, aryl, and heterocyclyl groups may be substituted with one or more of carbocyclyl, cycloalkyl; cycloalkenyl; aryl; heterocyclyl; heteroaryl; halo; OH; cyano; alkoxy; alkenyloxy; thioalkyl; NO₂; N₃; NRR; wherein each of R and R′ is, independently, H, alkyl, alkenyl, alkynyl, cycloalkyl, cycloalkenyl, aryl, or heterocyclyl; SO₂R, wherein R is H, alkyl or aryl; SO₂NRR′, wherein each of R and R′ is, independently, H, alkyl, or aryl; or NRSO₂R, wherein each of R and R′ is, independently, H, alkyl, alkenyl, alkynyl, cycloalkyl, cycloalkenyl, aryl, or heterocyclyl e. Aryl, carbocyclyl, cycloalkyl, cycloalkenyl, heteroaryl, and heterocyclyl groups may also be substituted with alkyl, alkenyl, or alkynyl. Alkyl, alkoxy, carbocyclyl, cycloalkyl, cycloalkenyl, and unsaturated heterocyclyl groups may also be substituted with oxo. In some embodiments, a substituent is further substituted as described herein. For example, a C₆ aryl group, i.e., phenyl, may be substituted with an alkyl group, which may be further substituted with a heterocyclyl group.

### DETAILED DESCRIPTION OF THE INVENTION

The present invention provides methods for the synthesis of small molecule complement factor D inhibitors and intermediates thereof. The complement factor D inhibitors are compounds of formula (XIV):

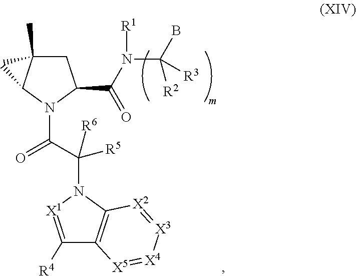

or pharmaceutically acceptable salts thereof, in which variables R¹-R⁶, X¹—X⁵, m, and B are as defined herein. Exemplary compounds of formula (XIV) are described in, e.g., U.S. Pat. Nos. 10,011,612; 10,287,301; and 10,822,352; and U.S. Patent Publication No. 2020/0071301 A1, the entire contents of which are incorporated herein by reference.

The methods include preparing a compound of formula (VIII):

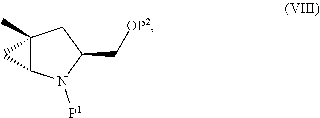

in which P¹ is H or an N-protecting group and P² is H or a hydroxyl protecting group (e.g., an ester hydroxyl protecting group such as benzoyl). The process involves providing a compound of formula (VII):

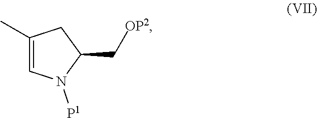

in which P¹ is H or an N-protecting group and P² is H or a hydroxyl protecting group (e.g., an ester hydroxyl protecting group such as benzoyl); and forming the compound of formula (VIII) from the compound of formula (VII) via a cyclopropanation reaction performed under Simmons-Smith reaction conditions. The Simmons-Smith reaction involves the formation of an organozinc carbenoid (e.g., iododomethyl zinc iodide formed from the reaction between Zn/Cu and diiodomethane) that reacts with an alkene to form a cyclopropane. Well-known modifications to the Simmons-Smith reaction includes the Furukawa Modification, in which Zn/Cu is replaced with diethyl zinc; the Charette Modification, in which diethylzinc is replaced with an aryldiazo compound such as phenyldiazomethane; and other modifications in which non-zinc agents, e.g., Sm/Hg and i-Bu₃Al. In some embodiments, the compound of formula (VII) is reacted with diethyl zinc and chloroiodomethane to form the compound of formula (VIII).

Embodiments of the method disclosed herein (e.g., wherein P² is benzoyl) unexpectedly provides the compound of (VIII) with high stereoselectivity (˜95:5), which is carried over in, e.g., subsequent reactions in the multi-step synthesis of the compounds of formula (XIV).

Previously reported procedures for preparing the compounds of formula (XIV) require the use of a compound of formula (I) which, prior to the disclosure, was typically prepared using a procedure reported in US Publication No. 2011/0274648 A1, which provides a ˜1:3 diastereomeric mixture of the compound of formula (I) and its diastereomer. Other published procedures used a silyl protective groups (Bioorg. Med. Chem., 21 (2013) 5725-5737), which render the resulting mixture difficult to purify, compared to the reaction disclosed herein. Another published procedure relies on lithium diisopropyl-amide and methyl iodide to insert a methyl group (see Formulae III to V), which results in a mixture of methylated compounds (both mono- and di-methylated) and unreacted starting material, requiring additional chromatographic separation.

The ability to prepare intermediates in the synthesis of the compound of (VIII) with high stereoselectivity substantially improves overall yield of the overall process for preparing the compounds of formula (XIV).

### Compounds of Formula (VII)

In some embodiments, the compound of formula (VII):

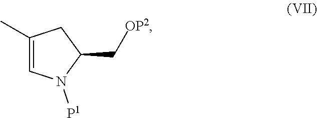

in which P¹ and P² are as defined above, is prepared by subjecting a compound of formula (VI):

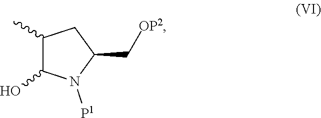

in which P¹ is an N-protecting group and P² is a hydroxyl protecting group (e.g., an ester hydroxyl protecting group such as benzoyl), to a dehydration reaction (an elimination reaction). The dehydration reaction is typically performed under elevated temperatures (in the presence of a strong acid, such as sulfuric acid, phosphoric acid, or trifluoracetic acid (e.g., formed from the hydrolysis of trifluoroacetic anhydride). The dehydration may also be performed in refluxing methylene chloride in the presence of catalytic p-toluenesulfonyl chloride (TsCl). Such reactions are well-known in the art. In some embodiments, the compound of formula (VII) is prepared by reacting the compound of formula (VI) with trifluoroacetic anhydride (e.g., in the presence of 2,6-lutidine).

### Compounds of Formula (VI)

In some embodiments, the compound of formula (VI):

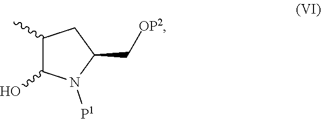

in which P¹ and P² are as defined above, is prepared by reducing a compound of formula (V):

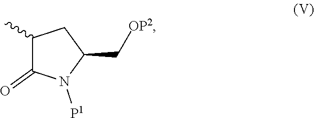

in which P¹ is an N-protecting group and P² is a hydroxyl protecting group (e.g., an ester hydroxyl protecting group such as benzoyl). The compound of formula (V) may be reduced using reducing agents including, but are not limited to, lithium triethylborohydride (“super hydride”) and sodium borohydride. In some embodiments, the compound of formula (V) is reduced with lithium triethylborohydride.

### Compounds of Formula (V)

In some embodiments, the compound of formula (V):

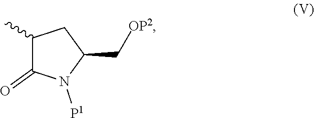

in which P¹ and P² are as defined above, is prepared by subjecting a compound of formula (IV):

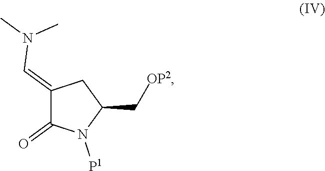

in which P¹ is H or an N-protecting group and P² is H or a hydroxyl protecting group (e.g., an ester hydroxyl protecting group such as benzoyl), to a hydrogenolysis reaction in the presence of a hydrogenation catalyst. Suitable hydrogenation catalysts include, but are not limited to, palladium on carbon, platinum(IV) oxide, palladium(II) hydroxide, Raney-Ni, and platinum metal. In some embodiments, the hydrogenolysis reaction is performed in the presence of palladium on carbon.

### Compounds of Formulas (IIA), (IIB), (Ill), and (IV)

In some embodiments, the compound of formula (IV):

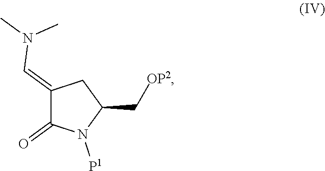

in which P¹ and P² are as defined above, is prepared by reacting a compound of formula (III):

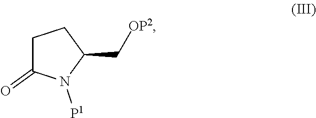

in which P¹ is an N-protecting group and P² is a hydroxyl protecting group (e.g., an ester hydroxyl protecting group such as benzoyl), with Bredereck's reagent (tert-butoxy bis(dimethylamino)methane; see, e.g., Rosso, Synlett. 2006; 5: 0809-0810). The compound of formula (III), in turn, may be prepared from (S)-5-(hydroxymethyl)pyrrolidin-2-one (commercially available) by protecting the hydroxyl group and the pyrrolidine nitrogen thereof with a hydroxyl protecting reagent and an N-protecting reagent, respectively. The hydroxyl group and the pyrrolidine may be protected in either order. That is, the hydroxyl group may be first protected to provide a compound of formula (IIA):

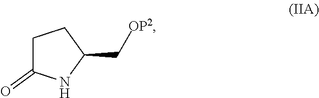

in which P² is a hydroxyl protecting group (e.g., an ester hydroxyl protecting group such as benzoyl), or the pyrrolidine nitrogen may be first protected to provide a compound (IIB):

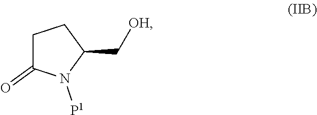

in which P¹ is an N-protecting group. The unprotected pyrrolidine nitrogen or hydroxyl group may be subsequently protected to provide a compound of formula (III). In some embodiments, the N-protecting reagent is (Boc)₂O and P¹ is Boc. In some embodiments, the hydroxyl protecting reagent is benzoyl chloride, and P² is benzoyl.

### Compounds of Formulas (IX) and (1)

In some embodiments, the compound of formula (VIII):

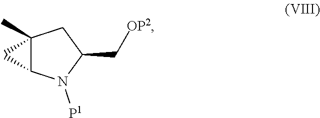

in which P¹ and P² are as defined above, is reacted with a hydroxyl protecting group removing agent to provide a compound of formula (IX):

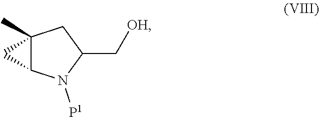

in which P¹ is H or an N-protecting group. The compound of formula (IX) may be further converted into a compound a compound of formula (I):

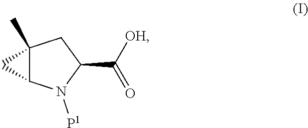

in which P¹ is H or an N-protecting group, by oxidation of the primary alcohol into a carboxylic acid. Methods for oxidizing primary alcohols into carboxylic acids are well known in the art. In some embodiments, the compound of formula (IX) is oxidized in the presence of (2,2,6,6-tetramethylpiperidin-1-yl)oxyl, sodium hypochlorite, and sodium chlorite. The compound of formula (IX) may also be oxidized under Jones oxidation conditions (i.e., in the presence of chromium trioxide dissolved in aqueous sulfuric acid), or with potassium permanganate (KMnO₄), pyridinium chlorochromate (PCC), tetrapropylammonium perruthenate (TPAP), or chromium trioxide/periodic acid (CrO₃—H₅IO₆).

In some embodiments, the compound of formula (I) is purified by first reacting it with an organic amine to form an organoammonium salt of the compound of formula (I) (e.g., in an organic solvent such as THE or toluene), then reacting the organoammonium salt of the compound of formula (I) with an acid to reform the compound of formula (I). Suitable organic amines include, but are not limited to, benzylamine and chiral amines such as alpha-methylbenzylamine. In some embodiments, the organic amine is benzylamine, which forms a benzylammonium salt of the compound of formula (I).

### Compounds of Formula (XI) and (XII)

In some embodiments, the compound of formula (I):

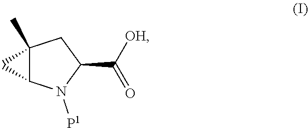

in which P¹ is an N-protecting group, is coupled to a compound of formula (X):

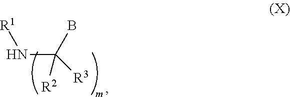

or a salt thereof, in which R¹ is H or optionally substituted C₁-C₆ alkyl; each of R² and R³ is independently H or methyl; m is 0, 1, or 2; and B is optionally substituted C₁-C₆ alkyl, optionally substituted C₂-C₆ alkenyl, optionally substituted C₃-C₁₀ carbocyclyl, optionally substituted C₆-C₁₄ aryl, or optionally substituted 5- to 10-membered heterocyclyl; in an amidation reaction to form a compound of formula (XI):

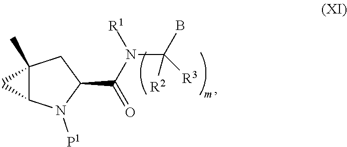

in which P¹ is an N-protecting group, and all other variables are as defined for formula (X). Alternatively, a compound of formula (I) in which P¹ is H may first be reacted with an N-protecting reagent before it is coupled to the compound of formula (X) or salt thereof. Subsequent removal of P¹ in the compound of formula (XI) with an N-protecting-group-removing agent provides a compound of formula (XII):

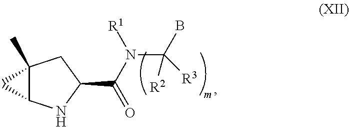

or a salt thereof, in which all variables are as defined for formula (XI).

In some embodiments, the N-protecting reagent is di-tert-butyl dicarbonate (Boc₂O), and the reaction is performed in an organic solvent (e.g., acetonitrile) in the presence of a base (e.g., 4-dimethylaminipyridine), and the N-protecting group is tert-butylcarbonate (Boc). In some embodiments in which the N-protecting group is Boc, the deprotection reaction includes treating the compound of formula (XI) with an acid in an organic solvent. In some embodiments, the acid is hydrogen chloride (4 N HCl in dioxane), and the reaction is performed in, e.g., dioxane. In some embodiments, the acid is hydrogen bromide (e.g., 33% HBr solution in acetic acid), and the reaction is performed in, e.g., ethyl acetate. In some embodiments, the acid is trifluoroacetic acid, and the reaction is performed in, e.g., dichloromethane. Other suitable N-protecting reagents and reaction conditions required to install and remove N-protecting groups are well known in the art (see, e.g., Wuts, Greene's Protective Groups in Organic Synthesis, Wiley-Interscience, 4th Edition, 2006).

In some embodiments, the compound of formula (I) and the compound of formula (X) or salt thereof are coupled in an organic solvent in the presence of a base and a coupling reagent. In some embodiments, the organic solvent is dimethylformamide. In some embodiments, the base is diisopropylethylamine. In some embodiments, the coupling reagent is (1-[bis(dimethylamino)methylene]-1H-1,2,3-triazolo[4,5-b]pyridinium 3-oxide hexafluorophosphate (HATU).

The compounds of formulas (XI) and (XII) can be prepared according to methods described in, e.g., U.S. Pat. Nos. 10,011,612, 10,287,301, and 10,822,352 and U.S. Patent Publications No. 2020/0071301 A1, the entire contents of which are incorporated herein by reference.

### Compounds of Formula (XIV)

In some embodiments, the compound of formula (XII):

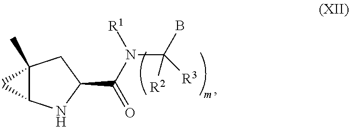

or the salt thereof is coupled to a compound of formula (XIII):

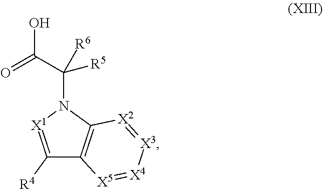

in which variables R⁴, R⁵, R⁶, X¹, X², X³, X⁴, and X⁵ are as defined herein, to form a compound of formula (XIV):

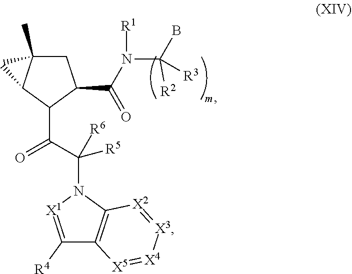

or a pharmaceutically acceptable salt thereof, in which all variables are as defined for formulas (XII) and (XIII). In some embodiments, the reaction is performed with a hydrochloride salt of the compound of formula (XII) and a compound of formula (XIII) in dimethylformamide in the presence of 1-[bis(dimethylamino)methylene]-1H-1,2,3-triazolo[4,5-b]pyridinium 3-oxide hexafluorophosphate and N,N-diisopropylethylamine. In some embodiments, the reaction is performed with a hydrobromide salt of the compound of formula (XII) and a compound of formula (XIII) in acetonitrile in the presence of propanephosphonic acid anhydride and N,N-diisopropylethylamine. In some embodiments, the reaction is performed with a trifluoroacetic acid salt of the compound of formula (XII) with a compound of formula (XIII) in dimethylformamide in the presence of N,N-diisopropylethylamine and 1-[bis(dimethylamino)methylene]-1H-1,2,3-triazolo[4,5-b]pyridinium 3-oxide hexafluorophosphate or 2-(1H-benzotriazole-1-yl)-1,1,3,3-tetramethylaminium tetrafluoroborate. In some embodiments, the reaction is performed with a salt (e.g., a hydrochloride salt, a hydrobromide salt, or a trifluoroacetic acid salt) of the compound of (XIII) and a compound of formula (XII).

Exemplary compounds of formulas (XII), (XIII), and (XIV) and their synthetic procedures are described in, e.g., U.S. Pat. Nos. 10,011,612, 10,287,301, and 10,822,352 and U.S. Patent Publications No. 2020/0071301 A1, the entire contents of which are incorporated herein by reference.

### EXAMPLES

The examples described herein serve to illustrate the present disclosure, and the disclosure is not limited to the examples given.

### Example 1: Synthesis of (1R,3S,5R)-2-(tert-butoxycarbonyl)-5-methyl-2-azabicyclo[3.1.0]hexane-3-carboxylic acid

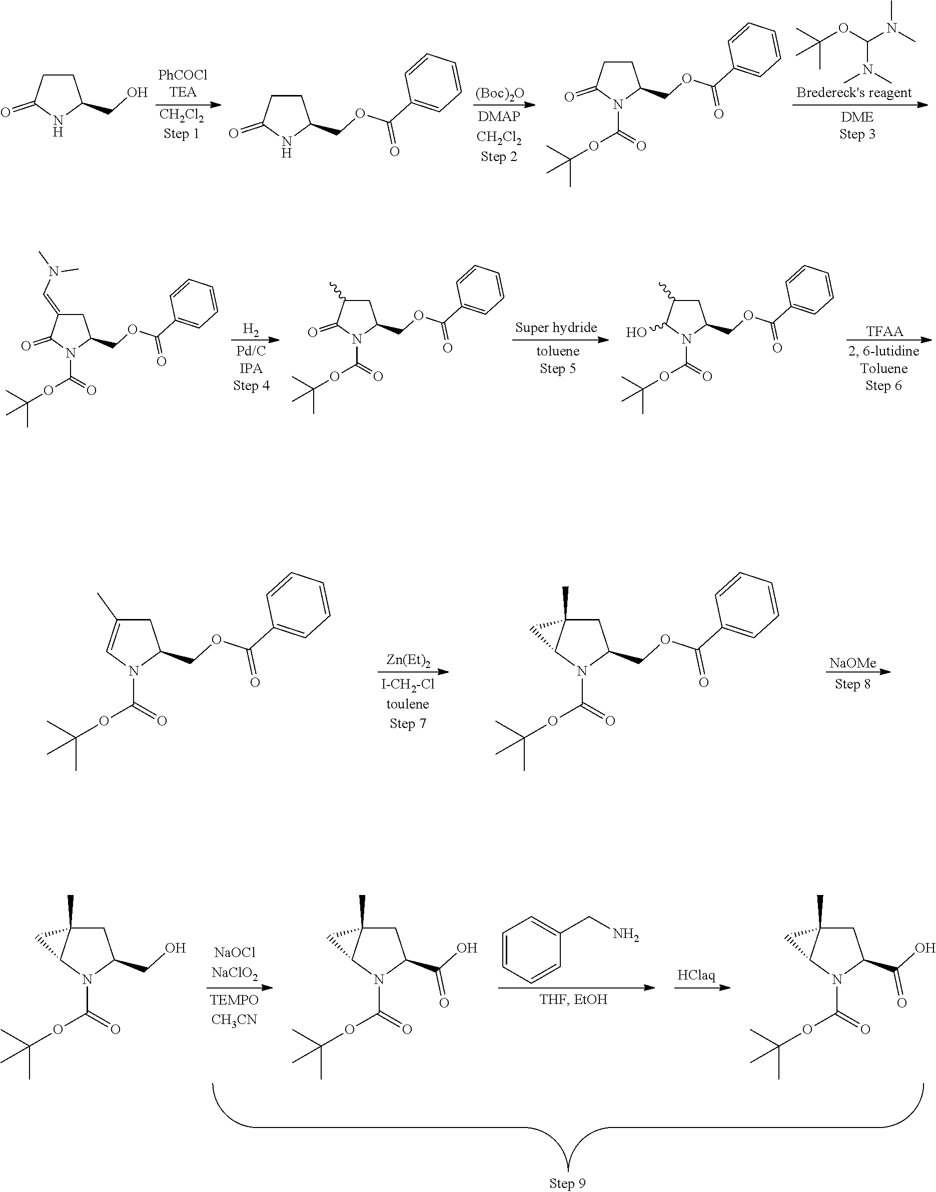

### Step 1: Synthesis of (S)-(5-oxopyroolidin-2-yl)methylbenzoate

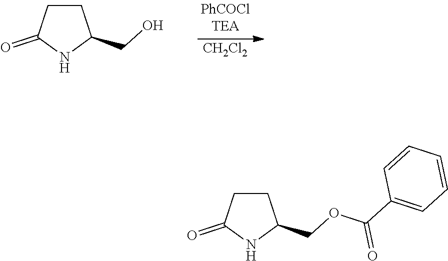

To the reactor added CH₂Cl₂ (1232 kg) at 25±5° C. under nitrogen atmosphere followed by (S)-5-(hydroxymethyl)-2-pyrrolidinone (154 kg). Then, the reaction mixture temperature was cooled to 0±5° C., and 4-(dimethylamino)pyridine (16.94 kg) was added. Triethylamine (158.62 kg) was added to the reaction mixture slowly, during which the reaction mixture was maintained at 0±5° C. Benzoyl chloride (190.96 kg) was subsequently added slowly to the reaction mixture was added at 0±5° C. under nitrogen atmosphere and stirred for 3 hr at same temperature. Next, 5% NaHCO₃ aq (˜46 Kg) was added, and the reaction mixture was warmed to 25±5° C. and stirred. The organic layer was separated and washed with 10% NaCl aq (770 kg) and concentrated under vacuum below 40° C., after which n-heptane (209 kg) was added to the reactor. The organic layer was then concentrated and cooled to 25±5° C. Then, 10% ethyl acetate in heptane (546.7 kg) was added to the reaction mixture, upon which (S)-(5-oxopyroolidin-2-yl)methylbenzoate was precipitated from solution and used directly in the next step.

### Step 2: Synthesis of tert-butyl (S)-2-((benzoyloxy)methyl)-5-oxopyrrolidine-1-carboxylate

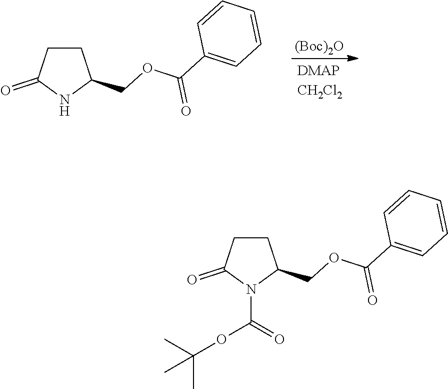 [SMILES: "(Boc)2O" ; O=C1CC[C@@H](COC(=O)c2ccccc2)N1 ; CC(C)(C)OC(=O)N1C(=O)CC[C@H]1COC(=O)c1ccccc1]

(S)-(5-oxopyroolidin-2-yl)methylbenzoate was collected by filtration and dissolved in CH₂Cl₂ (1001 kg), and 4-(dimethylamino)pyridine (72.38 kg) was added to the resulting solution at 25±5° C. Di-tert-butyl dicarbonate (495.88 kg) was charged into an addition vessel and added into the reaction mixture at 25±5° C. under nitrogen atmosphere. The reaction mixture was stirred for 1 hr at 25±5° C., after which water (1540 kg) was added, and the reaction mixture was stirred for an additional 10 min. The organic layer was separated, washed with 5% HCl (1725 kg) and NaCl aq. (770 kg), and concentrated under vacuum below 40° C. N-heptane (209 kg) was subsequently added the reactor, and the mixture was then concentrated under reduced pressure and cooled to 25±5° C. Then, 10% ethyl acetate in heptane (546.7 kg) was added to the mixture. The precipitate thus formed was collected by filtration and dried to afford the title compound (390 kg, 91.3% from step 1), which was characterized by LC-MS and compared to a reference sample.

### Step 3: Synthesis of tert-butyl (S,E)-5-((benzoyloxy)methyl)-3-((dimethylamino)methylene)-2-oxopyrrolidine-1-carboxylate

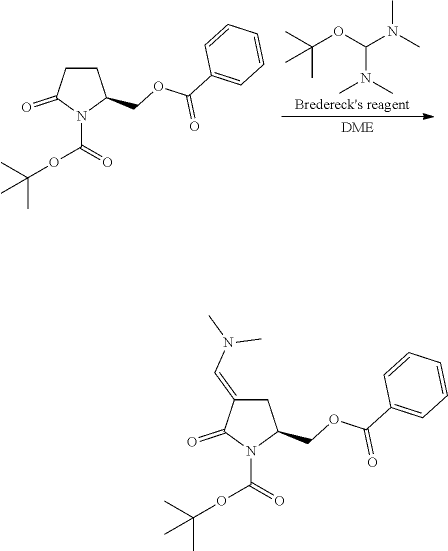

1,2-Dimethoxy ethane (234 kg) was charged into a reactor under nitrogen atmosphere, to which tert-butyl (S)-2-((benzoyloxy)methyl)-5-oxopyrrolidine-1-carboxylate (180 kg) was subsequently added. The reaction mixture was stirred at 25±5° C. for no less than 10 min, and tert-butyl-bis(dimethylamino)methane (147.6 kg) was added. The reaction temperature was raised to 75±5° C. and maintained until tert-butyl (S)-2-((benzoyloxy)methyl)-5-oxopyrrolidine-1-carboxylate was fully consumed as determined by reverse phase HPLC with a gradient elution. Then, the reaction temperature was cooled to 25±5° C., and ethyl acetate (810 kg) and water (900 kg) were added. The reaction mixture was stirred for 10 min, after which the organic layer was separated, and the solvent removed. Heptane (1476 Kg) was slowly added, and the mixture was stirred for 60 min, during which tert-butyl (S,E)-5-((benzoyloxy)methyl)-3-((dimethylamino)methylene)-2-oxopyrrolidine-1-carboxylate as a precipitate. The precipitate was collected by filtration, dried under vacuum, and used in the next step without purification.

### Step 4: Synthesis of tert-butyl (5S)-5-((benzoyloxy)methyl)-3-methyl-2-oxopyrrolidine-1-carboxylate

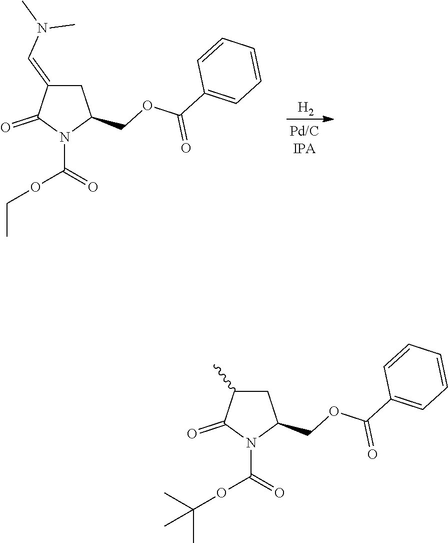 [SMILES: CCOC(=O)N1C(=O)/C(=C/N(C)C)C[C@H]1COC(=O)c1ccccc1 ; "H2" ; CC1C[C@@H](COC(=O)c2ccccc2)N(C(=O)OC(C)(C)C)C1=O]

Dried tert-butyl (S,E)-5-((benzoyloxy)methyl)-3-((dimethylamino)methylene)-2-oxopyrrolidine-1-carboxylate, isopropyl alcohol (IPA; 150 L), and palladium on carbon (10%-50% wet basis, 18 kg) was added to a hydrogenator. H₂ (4-5 atm) was then introduced into the hydrogenator, and the reaction mixture was heated to 60±5° C. and stirred for 20 h. The reaction mixture was filtered using celite, and the solvent was removed under reduced pressure with heating at ˜30-40° C. To the resulting residue was added 20% ethyl acetate in heptane (1750 kg), celite (36 kg), and silica gel (54 kg), and the mixture was stirred for 60 min at 25±5° C. The reaction mixture was filtered, and the solvent was removed under reduced pressure. The residue was redissolved in toluene (313 kg) and stirred for 10 min to obtain a solution of tert-butyl (5S)-5-((benzoyloxy)methyl)-3-methyl-2-oxopyrrolidine-1-carboxylate in toluene, which was used directly in the next step.

### Step 5: Synthesis of tert-butyl (5S)-5-((benzoyloxy)methyl)-2-hydroxy-3-methylpyrrolidine-1-carboxylate

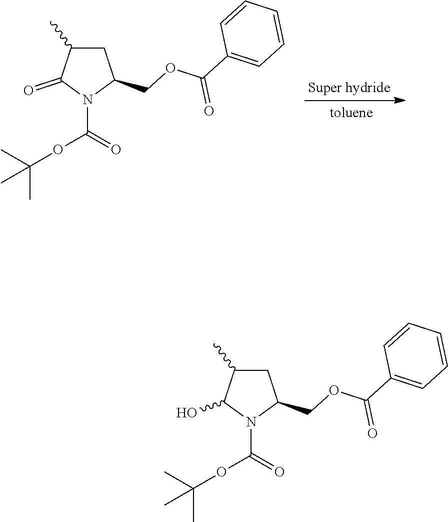

Toluene (1131 kg) was charged into a reactor, followed by the solution of tert-butyl (5S)-5-((benzoyloxy)methyl)-3-methyl-2-oxopyrrolidine-1-carboxylate (130 Kg) in toluene obtained from the previous step. Then, the solution was cooled to −65±5° C., and lithium triethylborohydride (20% in THF; superhydride) (225.5 Kg) was slowly added into the reactor, after which the reaction mixture was stirred for 1.5 hr at the same temperature. Acetic acid (28.6 kg) in toluene (452.4 kg) was then slowly added to the reaction mixture at ˜65±5° C., and the mixture was warmed to ˜10±5° C. Then, a ˜4% sodium hypochlorite solution (1079 kg) was added, and the reaction mixture was stirred for 15 min at ˜5±5° C. The organic layer was subsequently separated. A ˜4% sodium hypochlorite solution (1079 kg) was added to the organic layer, then the reaction mixture was stirred for 15 min at −5±5° C. The separation, addition of ˜4% sodium hypochlorite solution, and stirring was repeated one more time, after which the organic layer was again separated. The organic layer was washed with water (1300 kg) followed by 10% sodium chloride aq (715 kg), then concentrated under vacuum below 40° C. to approximately 10% w/v of the titled compound in solution, and reaction mass was cooled to 0±5° C.

### Step 6: Synthesis of tert-butyl (S)-2-((benzoyloxy)methyl)-4-methyl-2,3-dihydro-1H-pyrrole-1-carboxylate

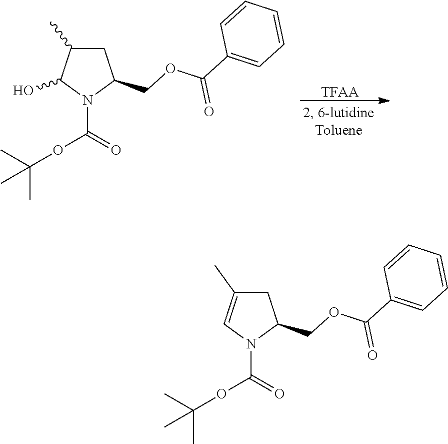

2,6-Lutidine (91 kg) was charged into the reactor containing the reaction mass obtained from the previous step at 0±5° C. Then, trifluoroacetic anhydride (89.7 kg) was charged slowly into the reactor at 0±5° C. The reaction temperature was raised to 50±5° C. and maintained at the same temperature until the tert-butyl (5S)-5-((benzoyloxy)methyl)-2-hydroxy-3-methylpyrrolidine-1-carboxylate was fully consumed, as confirmed by LC-MS. The reaction mixture was washed with 10% NaHCO₃ aq (650 kg), 0% citric acid aq (1430 kg), and 5% sodium chloride aq (617.5 kg), after which the organic layer concentrated was concentrated to obtain the compound in crude form, which was used directly in the next step.

### Step 7: Synthesis of tert-butyl (1R,3S,5R)-3-((benzoyloxy)methyl)-5-methyl-2-azabicyclo[3.1.0]hexane-2-carboxylate

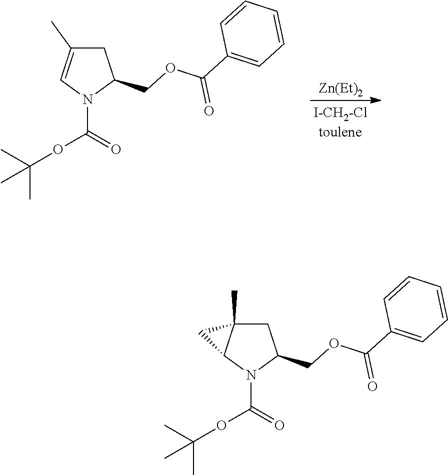

Toluene (2232 kg) was charged into a reactor at 25±5° C., to which tert-butyl (S)-2-((benzoyloxy)methyl)-4-methyl-2,3-dihydro-1H-pyrrole-1-carboxylate (124 kg) was added. The reaction mixture was cooled to −25±5° C., after which 1.5 M diethylzinc in toluene (595.5 kg) was slowly added under nitrogen atmosphere, followed by the gradual addition of chloroiodomethane (405.48 kg). The reaction was warmed to −2±5° C. and stirred for 12 h, after which a 10% NaHCO₃ aq solution (1116 kg) was added while maintaining the same temperature. The reaction mixture was then warmed to 25±5° C., and celite (37.2 kg) was charged into the reactor. After stirring for 10 min at 25±5° C., the mixture was filtered through a celite bed, and the layers were separated. The organic layer was washed with 5% sodium chloride aq (1240 kg), and then solvent was removed under reduced pressure with heating to obtain crude tert-butyl (1R,3S,5R)-3-((benzoyloxy)methyl)-5-methyl-2-azabicyclo[3.1.0]hexane-2-carboxylate (95:5 as determined by reverse phase HPLC with gradient elution), which was used directly in the next step.

### Step 8: Synthesis of tert-butyl (1R,3S,5R)-3-(hydroxymethyl)-5-methyl-2-azabicyclo[3.1.0]hexane-2-carboxylate

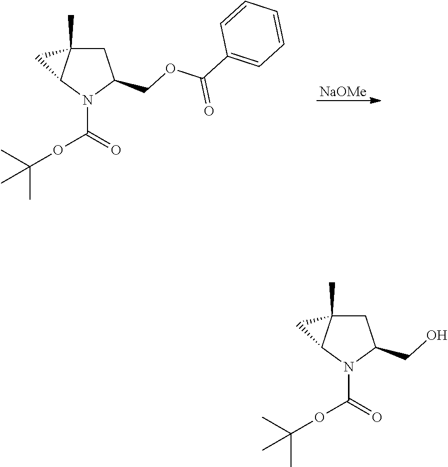

The crude tert-butyl (1R,3S,5R)-3-((benzoyloxy)methyl)-5-methyl-2-azabicyclo[3.1.0]hexane-2-carboxylate was dissolved in MeOH (979.6 kg), and the solution was cooled to 0±5° C. A 25% sodium methoxide solution (73.1 kg) was then slowly added to the reaction mixture at 0-5° C., and the mixture was stirred for 2 h. Water (1240 kg) was subsequently slowly added, and the reaction mixture was stirred at 25±5° C. for 8 h. Then, ethyl acetate (8.89 w/w) was charged into the reaction mixture, and the layers were separated. The organic layer was washed with 5% sodium chloride aq (620 kg) and concentrated under vacuum below 45° C. to obtain crude tert-butyl (1R,3S,5R)-3-(hydroxymethyl)-5-methyl-2-azabicyclo[3.1.0]hexane-2-carboxylate, which was used directly in the next step.

### Step 9: Synthesis of (1R,3S,5R)-2-(tert-butoxycarbonyl)-5-methyl-2-azabicyclo[3.1.0]hexane-3-carboxylic acid

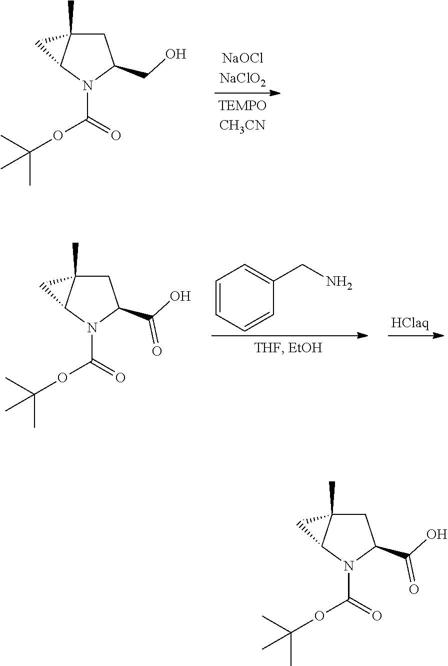

Acetonitrile (169.3 kg) and crude tert-butyl (1R,3S,5R)-3-(hydroxymethyl)-5-methyl-2-azabicyclo[3.1.0]hexane-2-carboxylate (42.96 kg) were charged into a reactor and stirred 5-10 min at 25±5° C., after which a sodium phosphate monobasic solution (107.4 kg in 232.0 kg water), TEMPO (3.01 kg), a sodium chlorite solution (12.89 kg, in 30.1 kg water) were added. The reaction mixture was stirred for 8 hours at below 35° C. Then, a sodium sulfite solution (171.84 kg in 859.2 kg water) was slowly added at 20±5° C. After the reaction mixture was stirred for 10 min, NaOH aq was added until the mixture reached pH 9-10, and the mixture was extracted with methyl tert-butyl ether (MTBE; 159.0 kg), and organic layer was discarded. The aqueous layer was adjusted to pH 2-3 with 2 M HCl aq, extracted with ethyl acetate (382.3 kg), and the organic layer thus obtained was washed with 5% sodium chloride aq (451.1 kg), isolated, and concentrated under vacuum below 55° C. THF (176.1 kg) and 2 M benzylamine in THF (21.48 kg benzylamine in 77.3 kg THF) were then added to at 15±5° C., then the reaction mixture was stirred at below 30° C. for 12 hr and then cooled to 0±5° C., during which the benzylammonium salt of (1R,3S,5R)-2-(tert-butoxycarbonyl)-5-methyl-2-azabicyclo[3.1.0]hexane-3-carboxylic acid precipitated from solution, which was collected by filtration. To the filtered solid was added HCl aq (98.8 kg in 859 kg water) and CH₂Cl₂ (257.8 Kg), after which the organic layer was washed with 5% sodium chloride aq (451.1 kg) and concentrated under vacuum below 45° C. Heptane was added to the resulting residue, after which a solid was formed. The solid was collected by filtration and dried under vacuum below 35° C. to afford the title compound 18.32 kg (40.2%). ¹H NMR (400 MHz, DMSO-d₆) δ 12.42 (s, 1H), 3.85 (t, 1H), 3.06 (m, 1H), 2.40 (m, 1H), 1.85 (m, 1H), 1.36 (m, 9H), 1.17 (s, 3H), 0.63 (m, 2H). HPLC: R.T. 19.039; 95% purity. Chiral HPLC: R.T. 8.964 min; 99.7% purity.

Reverse phase HPLC with a gradient program and DAD/VWD detection technique was performed using an Agilent 1200/1260 HPLC series system with DAD/VWD (or equivalent) equipped with an Atlantis T3 (150×4.6 mm, 3 μm) at column temperature 40° C. under the following conditions:

| | Flow Rate | 0.01% Perchloric Acid | |
|---|---|---|---|
| Time (min) | (mL/min) | in Water | Aceonitrile |
| 0 | 1.0 | 97 | 3 |
| 5 | 1.0 | 97 | 3 |
| 15 | 1.0 | 60 | 40 |
| 25 | 1.0 | 10 | 90 |
| 35 | 1.0 | 10 | 90 |
| 35.1 | 1.0 | 97 | 3 |
| 40 | 1.0 | 97 | 3 |

The UV detection was performed at 210 nm.

Chiral HPLC was performed using a CHIRALPAK® IG-3, 250×4.6 mm, 3 μm column with a column temperature 40° C. The isocratic mobile phase was formed by mixing 70% of trifluoroacetic acid (0.1%) in water and 30% acetonitrile and applied at flow rate of 0.8 mL/min for 30 min. The UV detection was performed at 210 nm. Samples were dissolved in methanol at 1 mg/ml. The injection volume was 5 μL.

### OTHER EMBODIMENTS

Various modifications and variations of the described compositions and methods of the disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosed methods that are obvious to those skilled in the art are intended to be within the scope of the disclosure.

Other embodiments are in the claims.

1. A method of preparing a compound of formula (VIII): 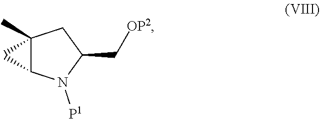
wherein P¹ is H or an N-protecting group and P² is H or a hydroxyl protecting group,
comprising: providing a compound of formula (VII): 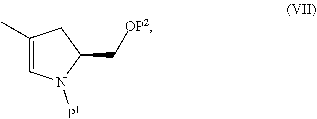
wherein P¹ is H or an N-protecting group and P² is H or a hydroxyl protecting group; and
forming the compound of formula (VIII) from the compound of formula (VII), said forming the compound of formula (VII) comprising reacting the compound of formula (II) under Simmons-Smith reaction conditions.

2. The method of claim 1, wherein said reacting the compound of formula (VII) under Simmons-Smith reaction conditions comprises reacting the compound of formula (VII) with diethylzinc and chloroiodomethane.

3. The method of claim 1 or 2, wherein said providing the compound of formula (VII) comprises:
providing a compound of formula (VI): 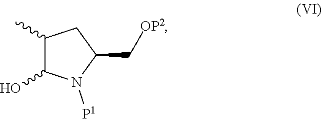
wherein P¹ is an N-protecting group and P² is a hydroxyl protecting group; and
subjecting the compound of formula (VI) to a dehydration reaction.

4. The method of claim 3, wherein the dehydration reaction comprises reacting the compound of formula (VI) with trifluoroacetic anhydride in the presence of 2,6-lutidine.

5. The method of claim of claim 3 and 4, wherein said providing the compound of formula (VI) comprises:
providing a compound of formula (V): 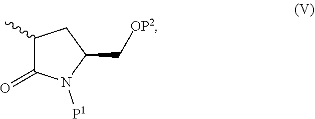
wherein P¹ is an N-protecting group and P² is a hydroxyl protecting group; and
reacting the compound of formula (V) with a reducing agent.

6. The method of claim 5, wherein the reducing agent is super hydride.

7. The method of claim 5 or 6, wherein said providing the compound of formula (V) comprises:
providing a compound of formula (IV): 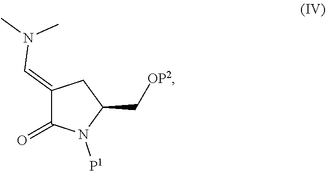
wherein P¹ is H or an N-protecting group and P² is H or a hydroxyl protecting group; and
subjecting the compound of formula (IV) to a hydrogenolysis reaction in the presence of a hydrogenation catalyst.

8. The method of claim 7, wherein the hydrogenation catalyst is palladium on carbon.

9. The method of claim 7 or 8, wherein said providing the compound of formula (IV) comprises:
providing a compound of formula (III): 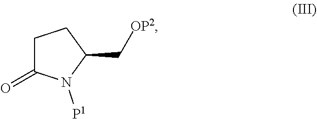
wherein P¹ is an N-protecting group and P² is a hydroxyl protecting group; and
reacting the compound of formula (III) with Bredereck's reagent.

10. The method of claim 9, wherein said providing the compound of formula (III) comprises:
providing a compound of formula (IIA): 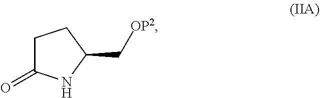
wherein P² is a hydroxyl protecting group; and
reacting the compound of formula (IIA) with an N-protecting reagent; or
providing a compound of formula (IIB): 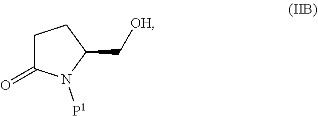
wherein P¹ is an N-protecting group; and
reacting the compound of formula (IIB) with a hydroxyl protecting reagent.

11. The method of claim 10, wherein said providing the compound of formula (III) comprises providing the compound of formula (IIA) and reacting it with an N-protecting reagent.

12. The method of claim 11, wherein said providing the compound of formula (IIA) comprises reacting (S)-5-(hydroxymethyl)pyrrolidin-2-one with a hydroxyl protecting reagent.

13. The method of claim 10, wherein said providing the compound of formula (III) comprises providing the compound of formula (IIB) and reacting it with a hydroxyl protecting reagent.

14. The method of claim 13, wherein said providing the compound of formula (IIB) comprises reacting (S)-5-(hydroxymethyl)pyrrolidin-2-one with an N-protecting reagent.

15. The method of any one of claims 1-14, wherein P² in formula (VIII) is a hydroxyl protecting group.

16. The method of claim 15, wherein the method further comprises reacting the compound of formula (VIII) with a hydroxyl protecting group removing agent to form a compound of formula (IX): 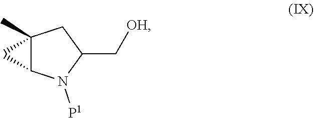
wherein P¹ is H or an N-protecting group.

17. The method of claim 16, wherein P¹ in formula (IX) is an N-protecting group.

18. The method of claim 17, wherein the method further comprises forming a compound of formula (I) from the compound of formula (IX), wherein the compound of formula (I) is of structure: 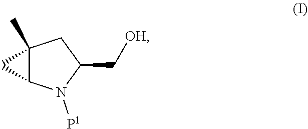
wherein P¹ is H or an N-protecting group; and
said forming the compound of formula (I) comprises oxidizing the compound of formula (IX).

19. The method of claim 18, wherein said oxidizing the compound of formula (IX) is performed in the presence of (2,2,6,6-tetramethylpiperidin-1-yl)oxyl, sodium hypochlorite, and sodium chlorite.

20. The method of claim 18 or 19, further comprising:
reacting the compound of formula (I) with an organic amine to form an organoammonium salt of the compound of formula (I);
reacting the organoammonium salt of the compound of formula (I) with an acid to form the compound of formula (I).

21. The method of claim 20, wherein the organic amine is benzylamine, and the organoammonium salt is a benzylammonium salt.

22. The method of any one of claims 18-21, wherein P¹ in formula (I) is an N-protecting group, and the method further comprises coupling the compound of formula (I) to a compound of formula (X): 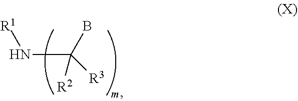
or a salt thereof, wherein
R¹ is H or optionally substituted C₁-C₆ alkyl;
each of R² and R³ is independently H or methyl;
m is 0, 1, or 2; and
B is optionally substituted C₁-C₆ alkyl, optionally substituted C₂-C₆ alkenyl, optionally substituted C₃-C₁₀ carbocyclyl, optionally substituted C₆-C₁₄ aryl, or optionally substituted 5- to 10-membered heterocyclyl;
to form a compound of formula (XI): 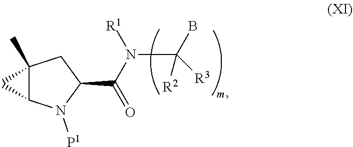
wherein
P¹ is an N-protecting group and all other variables are as defined for formula (X); and
reacting the compound of formula (XI) with a N-protecting-group-removing agent to form a compound of formula (XII): 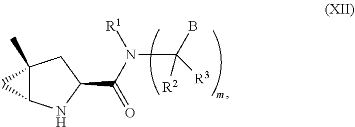
or a salt thereof, wherein all variables are as defined for formula (XI).

23. The method of claim 22, further comprising
coupling the compound of formula (XII) or the salt thereof to a compound of formula (XIII): 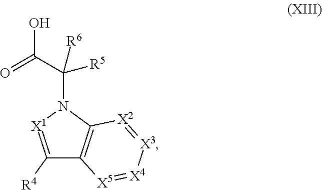
or a salt thereof, wherein
R⁴ is H; halo; OH; NH₂; cyano; optionally substituted C₁-C₆ alkyl; optionally substituted C₂-C₆ alkenyl; optionally substituted 3- to 8-membered heterocyclyl; —C(O)NRₐRₐ′, wherein each of Rₐ and Rₐ′ is, independently, H, optionally substituted C₁-C₆ alkyl, optionally substituted C₂-C₆ alkenyl, optionally substituted C₂-C₆ alkynyl, or optionally substituted C₃-C₈ cycloalkyl; —C(O)R_{b}; —OC(O)R_{b}; or —C(O)OR_{b}; wherein R_{b}, in each instance, is selected from H, optionally substituted C₁-C₆ alkyl, optionally substituted C₁-C₆ alkoxy, and optionally substituted C₃-C₈ carbocyclyl;
each of R⁵ and R⁶ is, independently, H or optionally substituted C₁-C₆ alkyl;
X¹ is N or CR_{c}, wherein R_{c} is H, halo, optionally substituted C₁-C₆ alkyl, or optionally substituted C₁-C₆ alkoxy;
each of X² and X⁵ is independently N or CR_{d}, wherein each R_{d} is independently selected from H, halo, cyano, optionally substituted C₁-C₆ alkyl, optionally substituted C₁-C₆ alkoxy, optionally substituted C₃-C₈ carbocyclyl, and optionally substituted 5- to 8-membered heteroaryl; and
each of X³ and X⁴ is independently selected from N, CRₑ, and CR_{f}, wherein Rₑ is selected from H, halo, cyano, optionally substituted C₁-C₆ alkyl, optionally substituted C₁-C₆ alkoxy, and —C(O)OR_{g}, wherein R_{g} is H or optionally substituted C₁-C₆ alkyl; and R_{f} is selected from optionally substituted C₄-C₁₀ aryl, optionally substituted 5- to 10-membered heteroaryl containing 1, 2, or 3 heteroatoms selected from N, O, and S, and optionally substituted 4- to 10-membered saturated or unsaturated non-aromatic heterocyclyl containing 1-4 heteroatoms selected from N, O, and S;
wherein at least one of X³ and X⁴ is CR_{f};
to form a compound of formula (XIV): 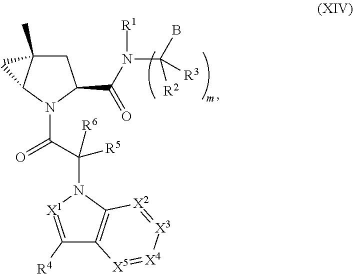
or a pharmaceutically acceptable salt thereof, wherein R¹, R², R³, m, and B are as defined for formula (XII), and all other variables are as defined for formula (XIII).

24. The method of claim 22 or 23, wherein P¹ is tert-butoxycarbonyl.

25. The method of claim 24, wherein the N-protecting-group-removing agent is hydrogen chloride, and said reacting the compound of formula (XI) with the N-protecting-group-removing agent forms a hydrochloride salt of the compound of formula (XII).

26. The method of claim 25, wherein the hydrochloride salt of the compound of formula (XII) is coupled to the compound of formula (XIII) in dimethylformamide in the presence of 1-[bis(dimethylamino)methylene]-1H-1,2,3-triazolo[4,5-b]pyridinium 3-oxide hexafluorophosphate and N,N-diisopropylethylamine.

27. The method of claim 22 or 23, wherein the N-protecting-group-removing agent is hydrogen bromide, and said reacting the compound of formula (XI) with the N-protecting-group-removing agent forms a hydrobromide salt of the compound of formula (XII).

28. The method of claim 27, wherein the hydrobromide salt of the compound of formula (XII) is coupled to the compound of formula (XIII) in acetonitrile in the presence of propanephosphonic acid anhydride and N,N-diisopropylethylamine.

29. The method of claim 22 or 23, wherein the N-protecting-group-removing agent is trifluoroacetic acid, and said reacting the compound of formula (XI) with the N-protecting-group-removing agent forms a trifluoroacetic acid salt of the compound of formula (XII).

30. The method of claim 29, wherein the trifluoroacetic acid salt of the compound of formula (I) is coupled to the compound of formula (XIII) in dimethylformamide in the presence of N, N-diisopropylethylamine and 1-[bis(dimethylamino)methylene]-1H-1,2,3-triazolo[4,5-b]pyridinium 3-oxide hexafluorophosphate or 2-(1H-benzotriazole-1-yl)-1,1,3,3-tetramethylaminium tetrafluoroborate.

31. The method of any one of claims 22-30, wherein R¹ is H.

32. The method of any one of claims 22-30, wherein R¹ is CH₃.

33. The method of any one of claims 22-32, wherein m is 1.

34. The method of any one of claims 22-32, wherein m is 2.

35. The method of any one of claims 22-32, wherein m is 0.

36. The method of any one of claims 22-35, wherein each of R² and R³ is H.

37. The method of any one of claims 22-35, wherein R² is H and R³ is CH₃.

38. The method of any one of claims 22-35, wherein each of R² and R³ is CH₃.

39. The method of any one of claims 22-38, wherein B is optionally substituted 5- to 10-membered heteroaryl.

40. The method of claim 39, wherein B is optionally substituted 6-membered heteroaryl.

41. The method of claim 40, wherein B is optionally substituted pyridyl, optionally substituted pyridazinyl, optionally substituted pyrimidinyl, or optionally substituted pyrazinyl.

42. The method of claim 41, wherein B is optionally substituted pyridyl.

43. The method of any one of claims 40-42, wherein B is:

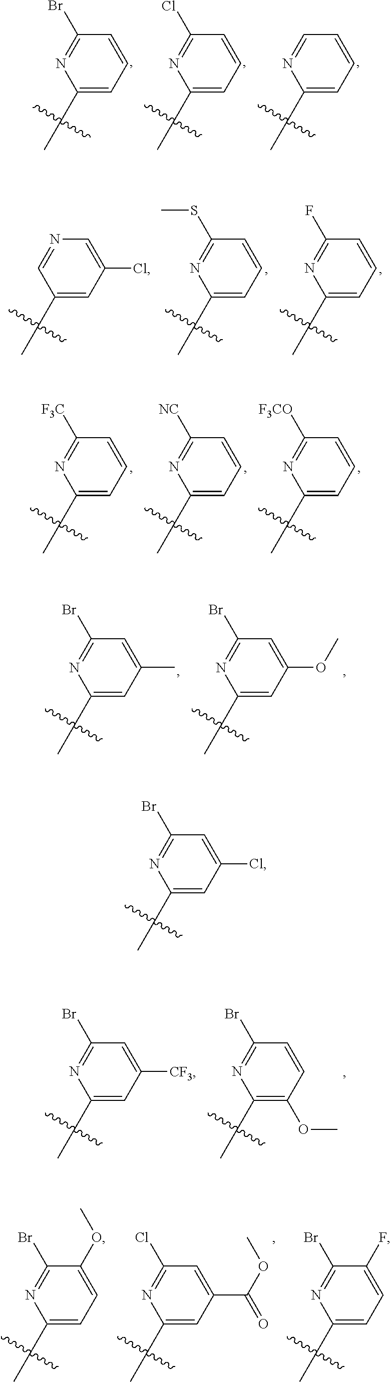

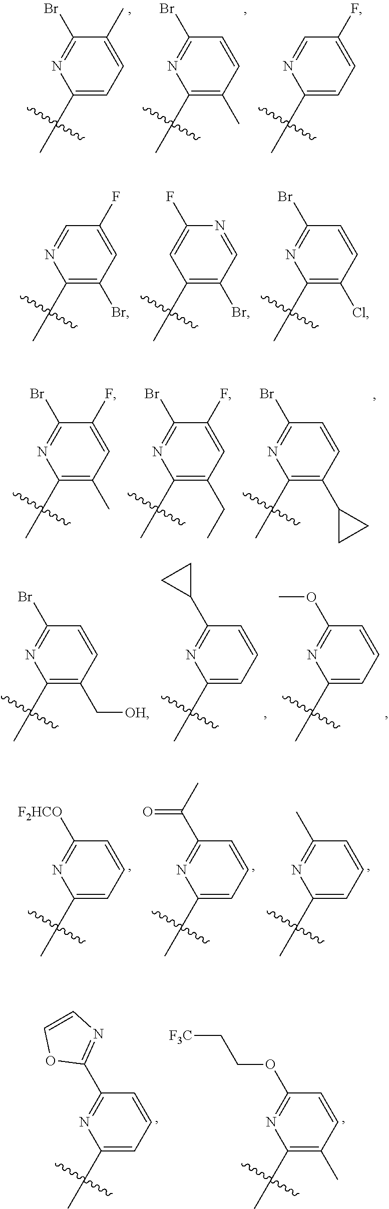

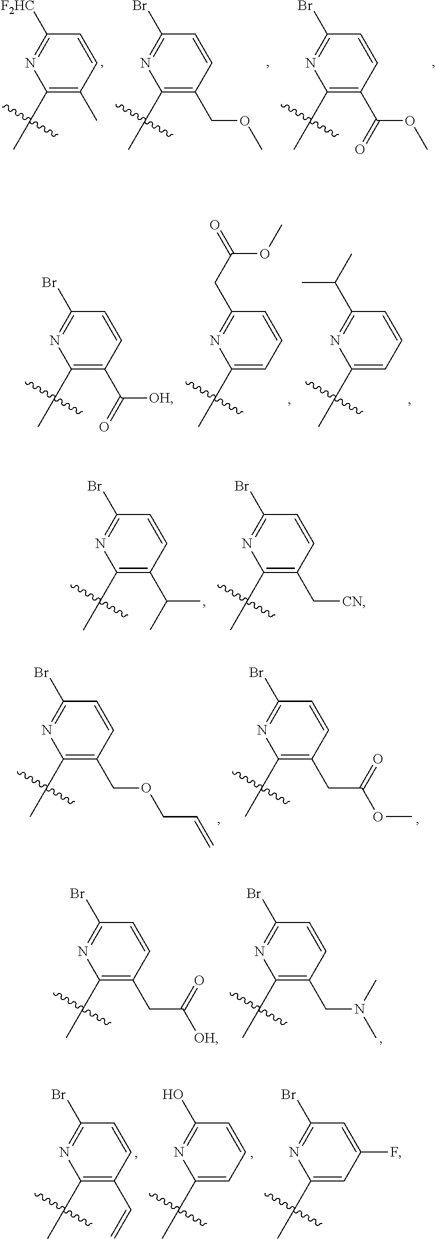

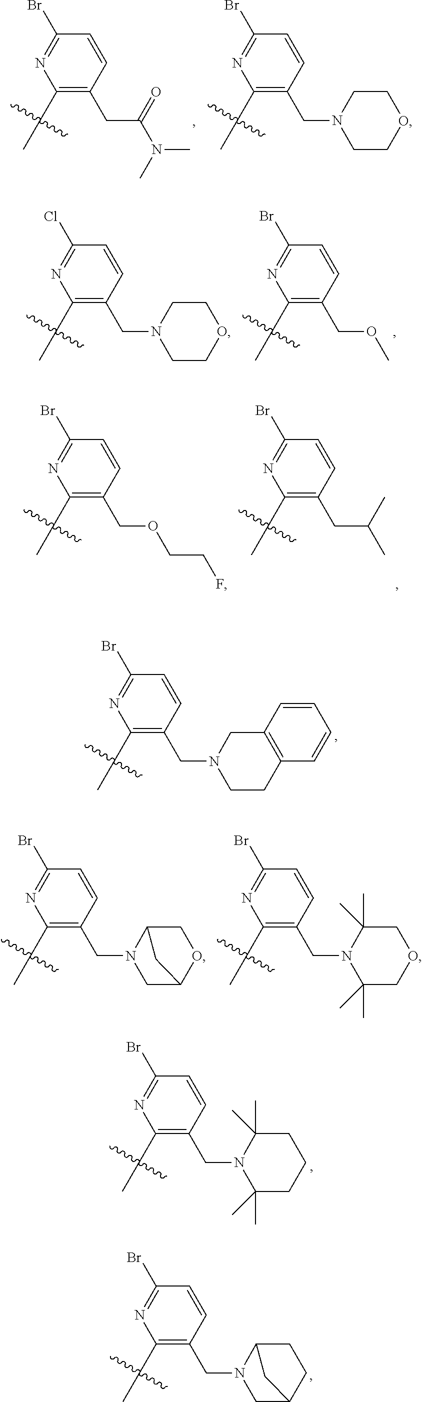

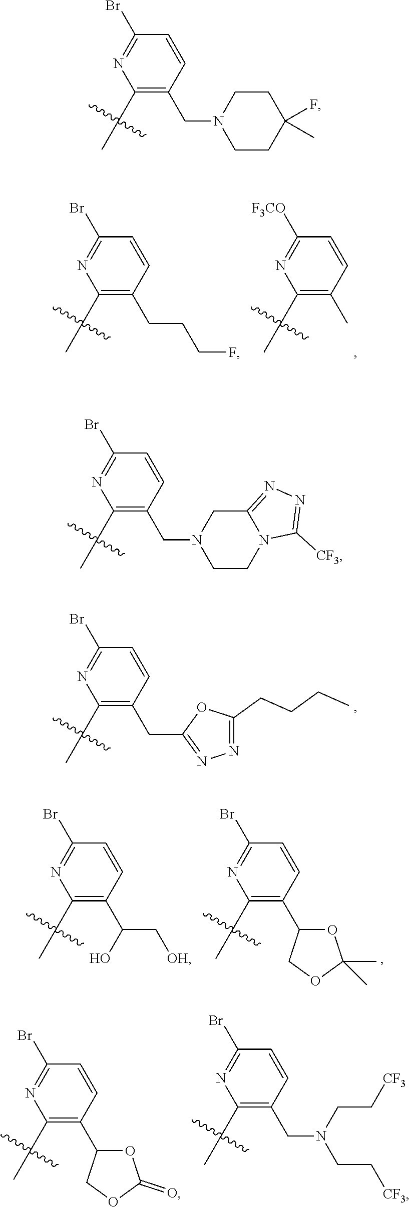

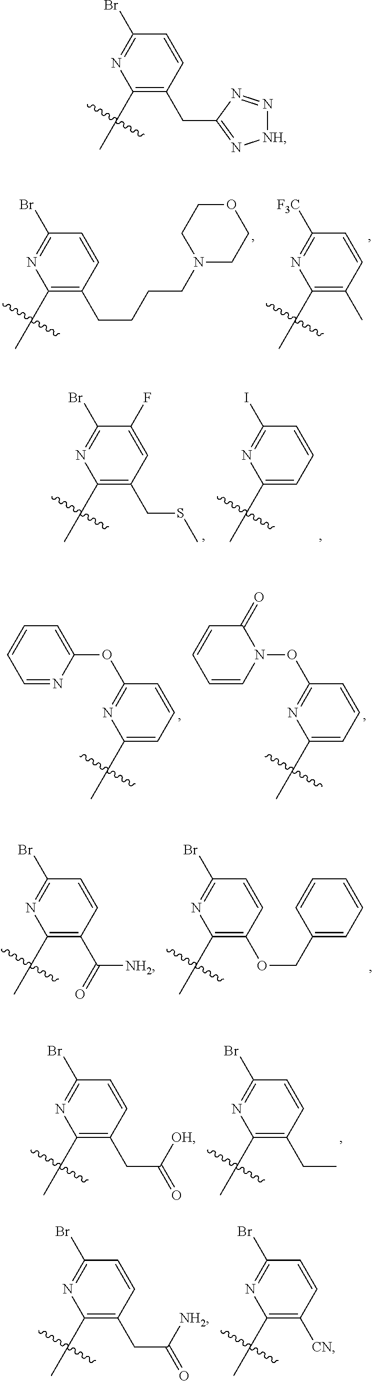

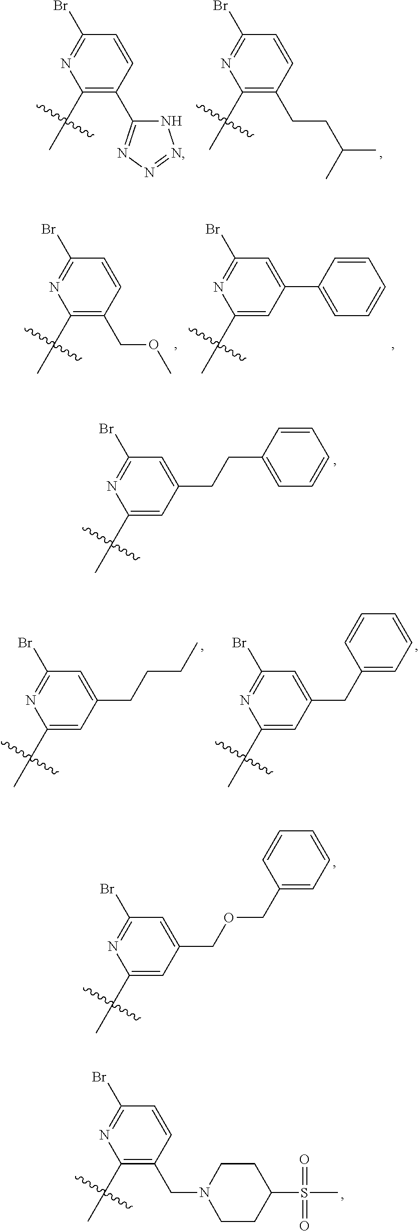

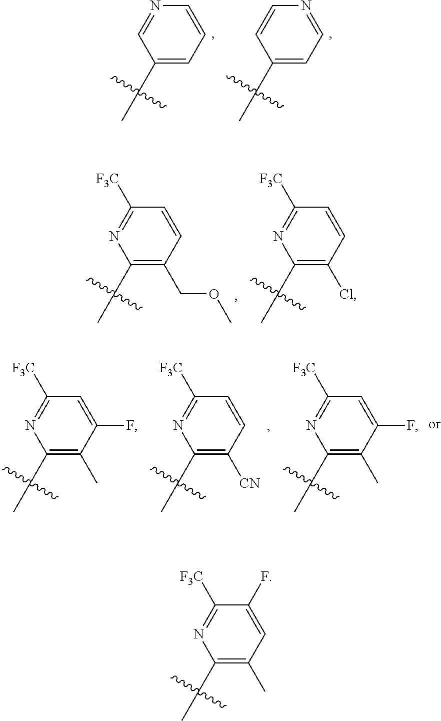

44. The method of claim 43, wherein B is

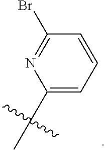 [SMILES: *c1cccc(Br)n1]

45. The method of claim 43, wherein B is

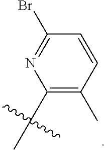 [SMILES: *c1nc(Br)ccc1C]

46. The method of claim 41, wherein B is optionally substituted pyrazinyl.

47. The method of claim 46, wherein B is:

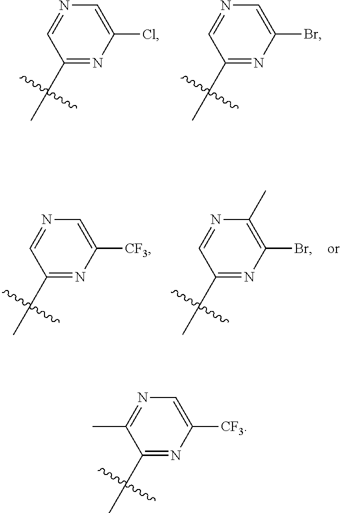

48. The method of claim 41, wherein B is optionally substituted pyrimidinyl.

49. The method of claim 48, wherein B is:

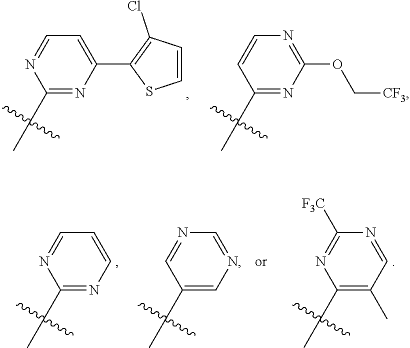 [SMILES: *c1nccc(-c2sccc2Cl)n1 ; *c1ccnc(OCC(F)(F)F)n1 ; *c1ncccn1 ; *c1cncnc1 ; *c1nc(C(F)(F)F)ncc1C]

50. The method of claim 41, wherein B is optionally substituted pyridazinyl.

51. The method of claim 50, wherein B is

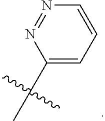 [SMILES: *c1cccnn1]

52. The method of claim 39, wherein B is optionally substituted five-membered heteroaryl.

53. The method of claim 52, wherein B is:

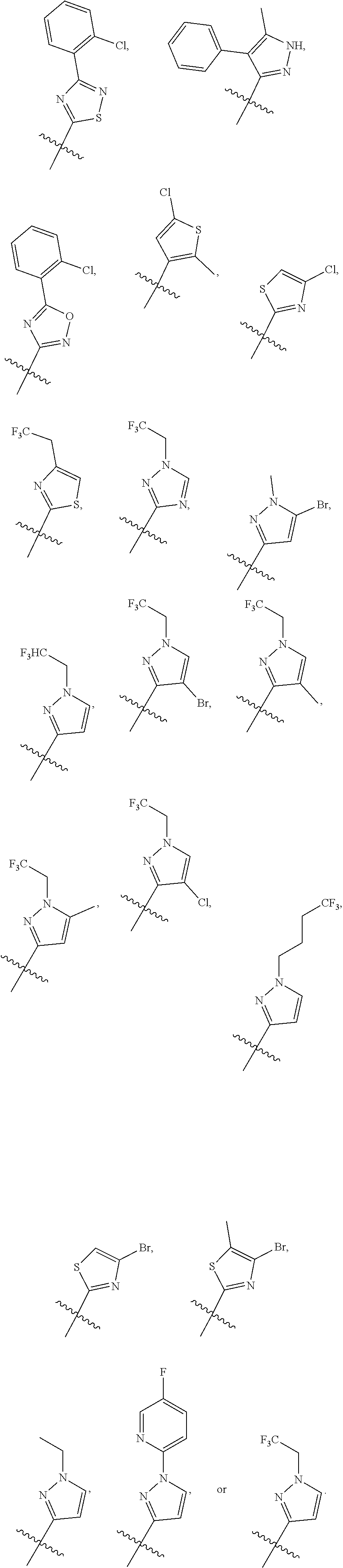

54. The method of claim 39, wherein B is bicyclic 9- or 10-membered bicyclic heteroaryl.

55. The method of claim 54, wherein B is

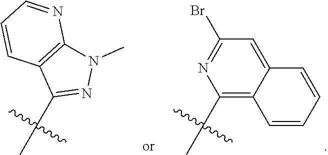 [SMILES: *c1nn(C)c2ncccc12 ; *c1nc(Br)cc2ccccc12]

56. The method of any one of claims 22-38, wherein B is optionally substituted C₆-C₁₄ aryl.

57. The method of claim 56, wherein B is optionally substituted phenyl.

58. The method of claim 57, wherein B is:

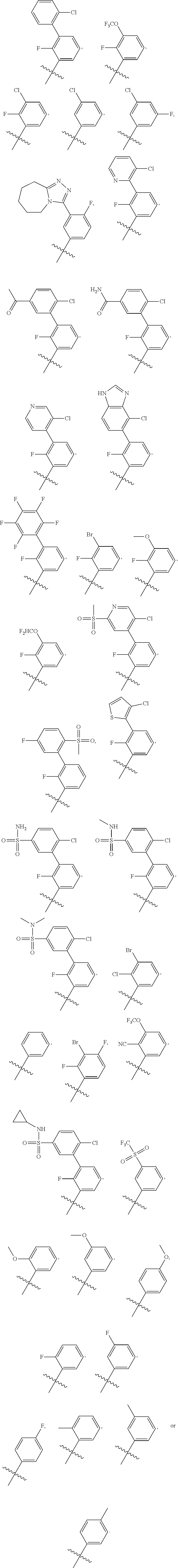

59. The method of any one of claims 22-38, wherein B is optionally substituted 5- to 9-membered unsaturated heterocyclyl.

60. The method of claim 59, wherein B is:

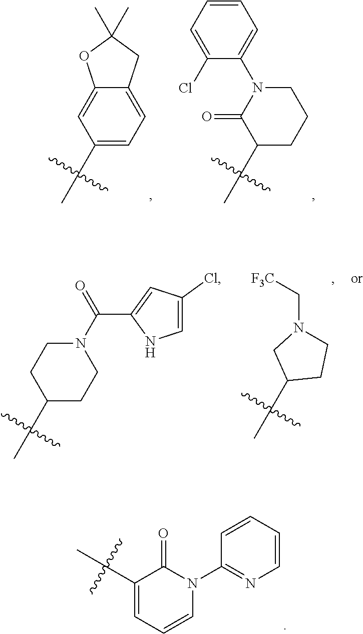 [SMILES: *c1ccc2c(c1)OC(C)(C)C2 ; *C1CCCN(c2ccccc2Cl)C1=O ; *C1CCN(C(=O)c2cc(Cl)c[nH]2)CC1 ; *C1CCN(CC(F)(F)F)C1 ; *c1cccn(-c2ccccn2)c1=O]

61. The method of any one of claims 22-38, wherein B is optionally substituted C₃-C₁₀ cycloalkyl.

62. The method of claim 61, wherein B is:

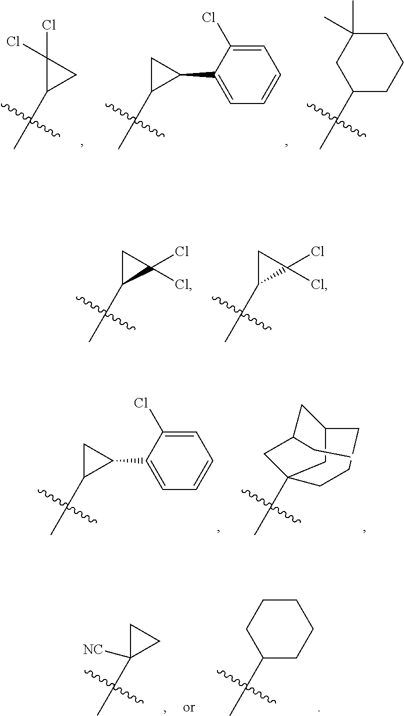

63. The method of any one of claims 22-38, wherein B is optionally substituted C₂-C₆ alkenyl.

64. The method of claim 63, wherein B is:

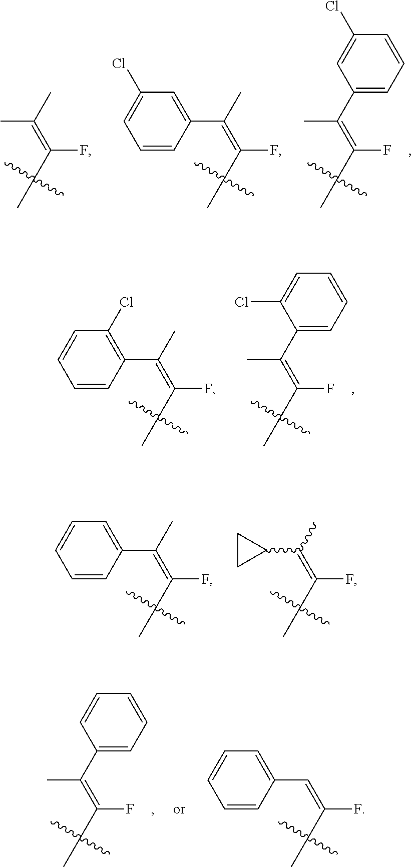

65. The method of any one of claims 22-38, wherein B is optionally substituted C₁-C₆ alkyl.

66. The method of claim 65, wherein B is:

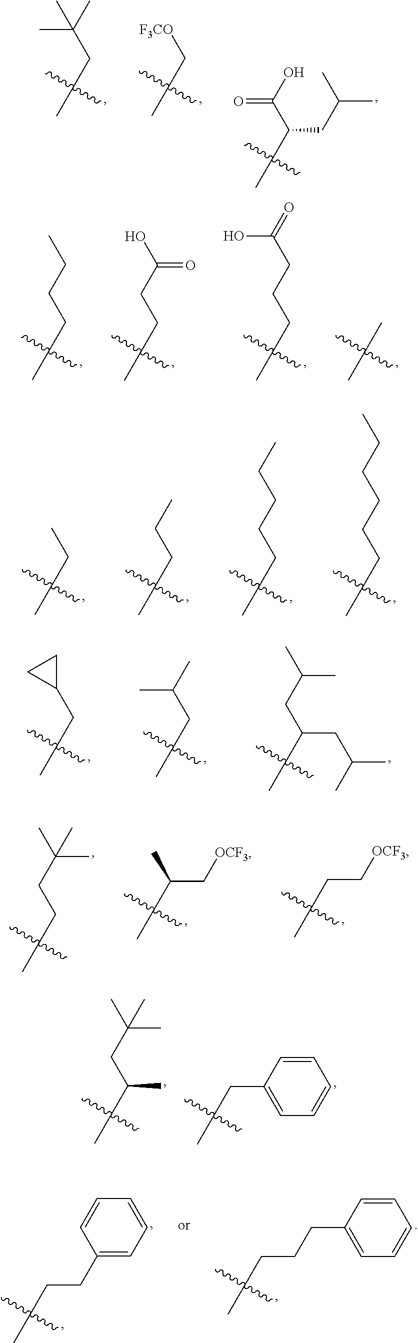

67. The method of any one of claims 23-66, wherein X¹ is N.

68. The method of any one of claims 23-66, wherein X¹ is CR_{c}.

69. The method of claim 68, wherein X¹ is C(CH₃).

70. The method of claim 68, wherein X¹ is CH.

71. The method of any one of claims 23-70, wherein X² is CR_{d}.

72. The method of claim 71, wherein R^{d} is H or optionally substituted C₁-C₆ alkyl.

73. The method of claim 71 or 72, wherein X² is CH.

74. The method of claim 71 or 72, wherein X² is C(CH₃).

75. The method of any one of claims 23-74, wherein X⁵ is CR_{d}.

76. The method of claim 75, wherein X⁵ is CH.

77. The method of any one of claims 23-76, wherein X³ is CR_{f}.

78. The method of claim 77, wherein X⁴ is N.

79. The method of claim 77, wherein X⁴ is CH.

80. The method of any one of claims 23-76, wherein X⁴ is CR_{f}.

81. The method of claim 80, wherein X³ is N.

82. The method of claim 80, wherein X³ is CH.

83. The method of any one of claims 23-82, wherein R_{f} is optionally substituted 5- to 10-membered heteroaryl containing 1, 2, or 3 heteroatoms selected from N, O, and S.

84. The method of claim 83, wherein R_{f} is 6-membered heteroaryl containing 1, 2, or 3 heteroatoms selected from N, O, and S.

85. The method of claim 84, wherein R_{f} is optionally substituted pyrimidinyl.

86. The method of claim 85, wherein R_{f} is:

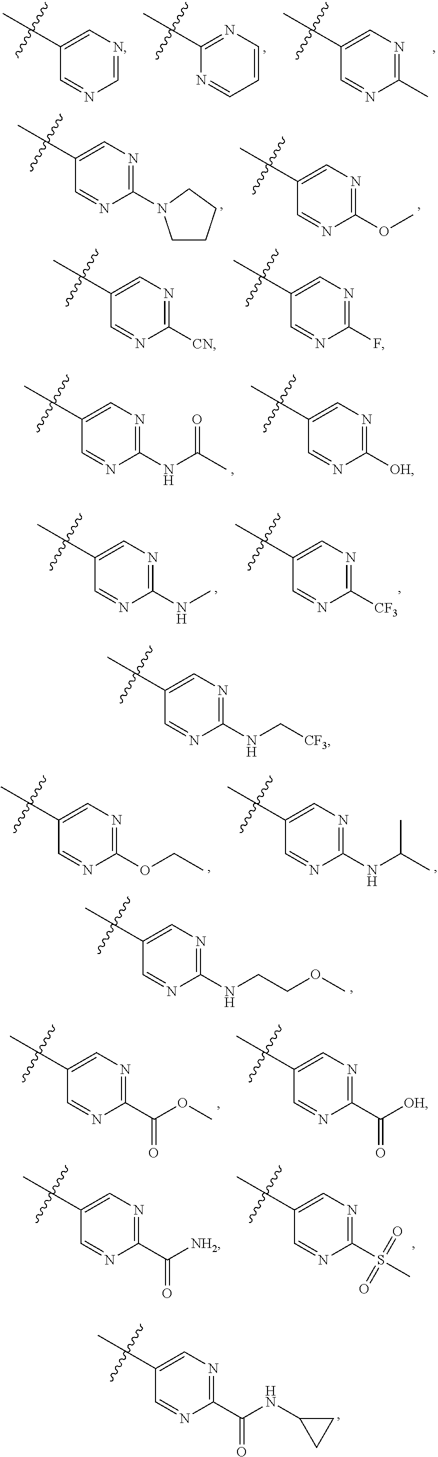

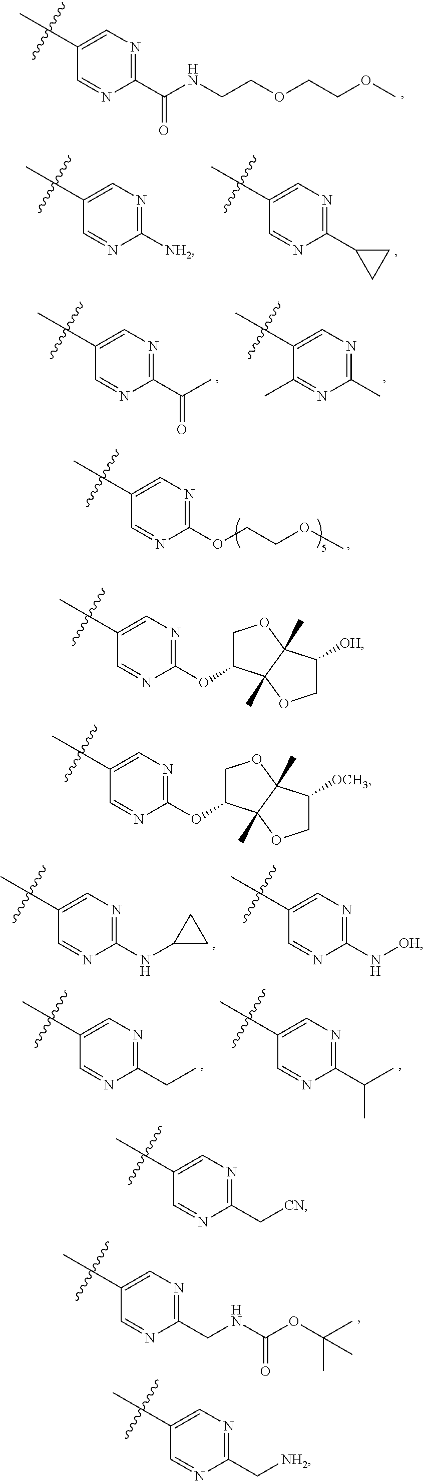

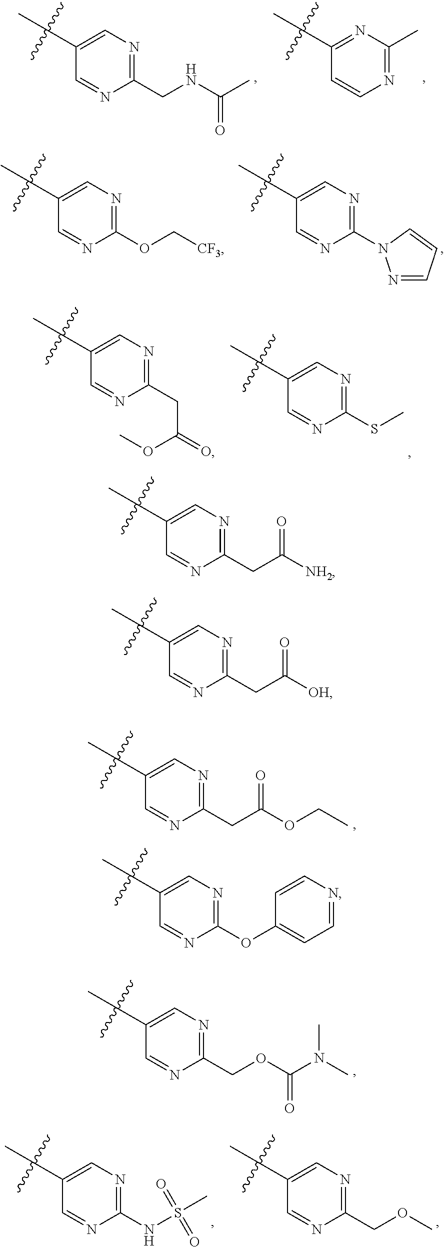

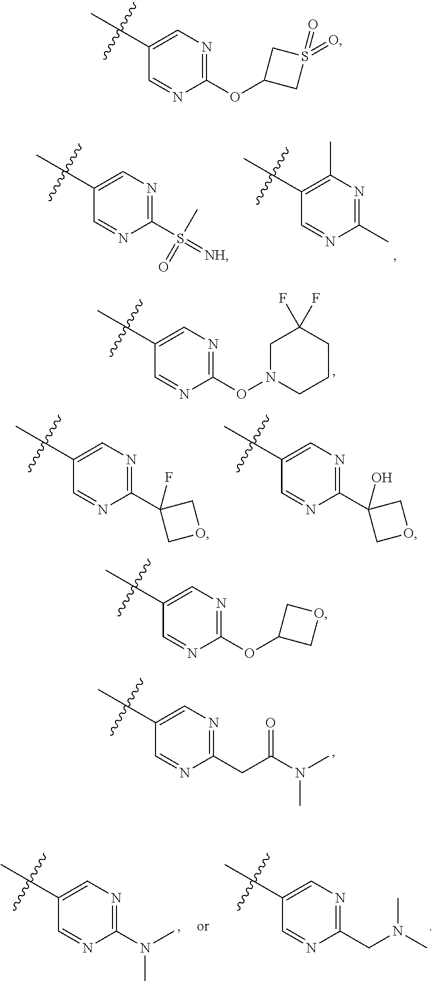

87. The method of claim 86, wherein R_{f} is

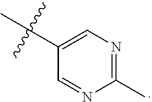 [SMILES: *c1cnc(C)nc1]

88. The method of claim 84, wherein R_{f} is

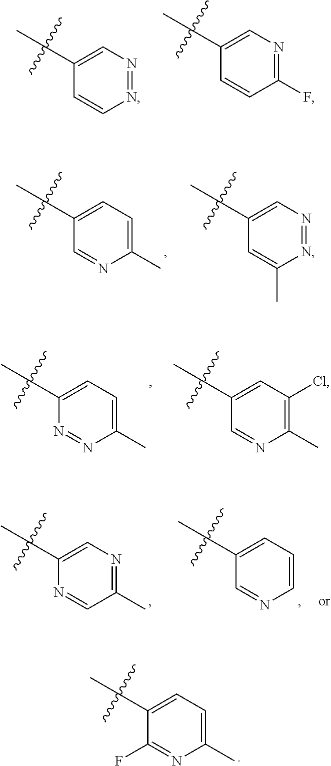

89. The method of claim 83, wherein R_{f} is optionally substituted 8- to 10-membered bicyclic heteroaryl containing 1, 2, or 3 heteroatoms selected from N, O, and S.

90. The method of claim 89, wherein R_{f} is optionally substituted pyrazolo[1,5-a]pyrimidinyl, optionally substituted [1,2,4]triazolo[1,5-a]pyridinyl, optionally substituted thiazolo[5,4-b]pyridinyl, optionally substituted imidazo[1,2-a]pyrimidinyl, optionally substituted 3H-imidazo[4,5-b]pyridinyl, 1H-thieno[3,2-c]pyrazolyl, imidazo[1,2-b]pyridazinyl, optionally substituted quinazolinyl, optionally substituted quinolinyl, and 1H-benzo[d]imidazolyl.

91. The method of claim 90, wherein R_{f} is:

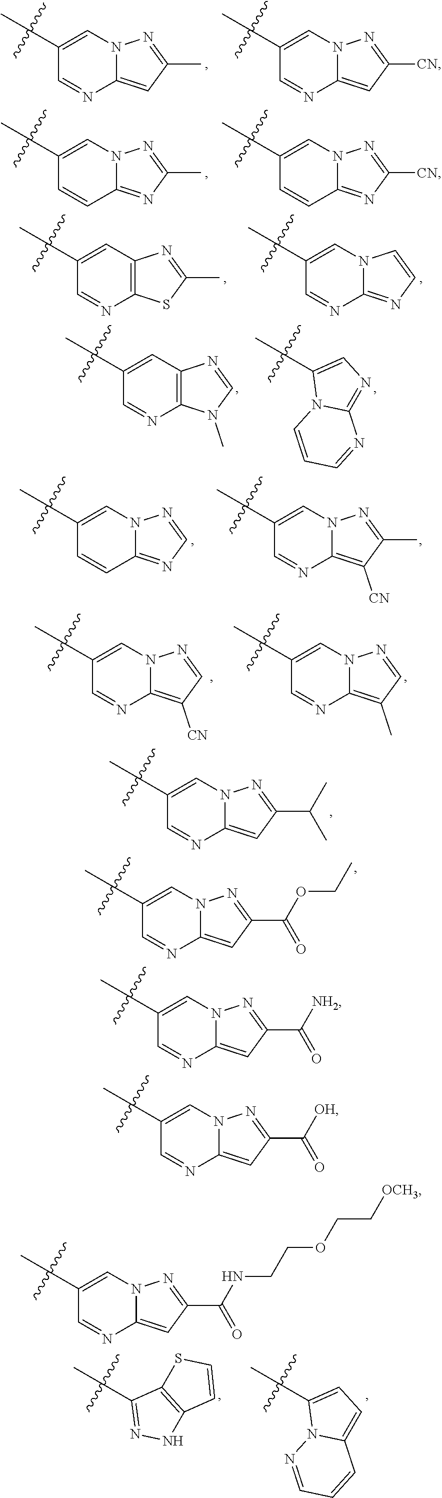

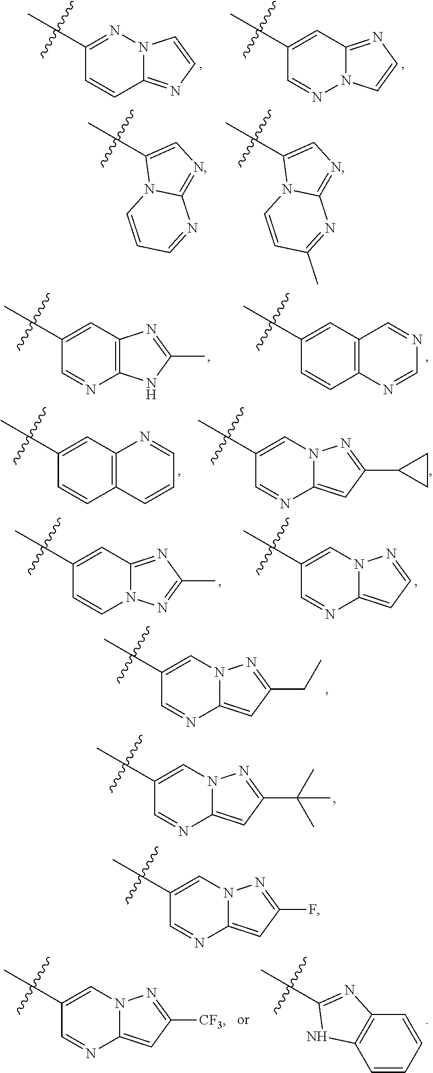

92. The method of claim 91, wherein R_{f} is

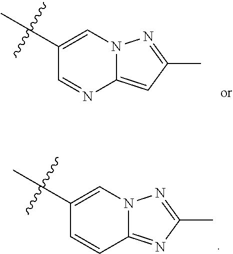 [SMILES: *c1cnc2cc(C)nn2c1 ; *c1ccc2nc(C)nn2c1]

93. The method of any one of claims 23-82, wherein R_{f} is optionally substituted C₆-C₁₄ aryl.

94. The method of claim 93, wherein R_{f} is optionally substituted phenyl.

95. The method of claim 94, wherein R_{f} is:

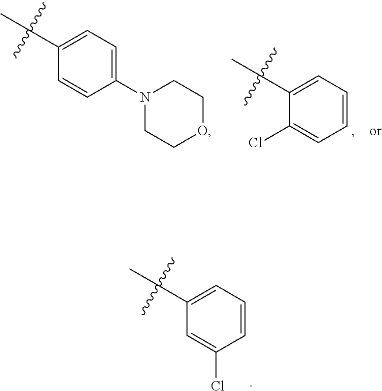 [SMILES: *c1ccc(N2CCOCC2)cc1 ; *c1ccccc1Cl ; *c1cccc(Cl)c1]

96. The method of any one of claims 23-82 wherein R_{f} is optionally substituted 6- to 9-membered unsaturated heterocyclyl containing 1-4 heteroatoms selected from N, O, or S.

97. The method of claim 96, wherein the R_{f} is bonded to the carbon atom to which it is attached through a carbon ring atom contained therein.

98. The method of claim 97, wherein R_{f} is:

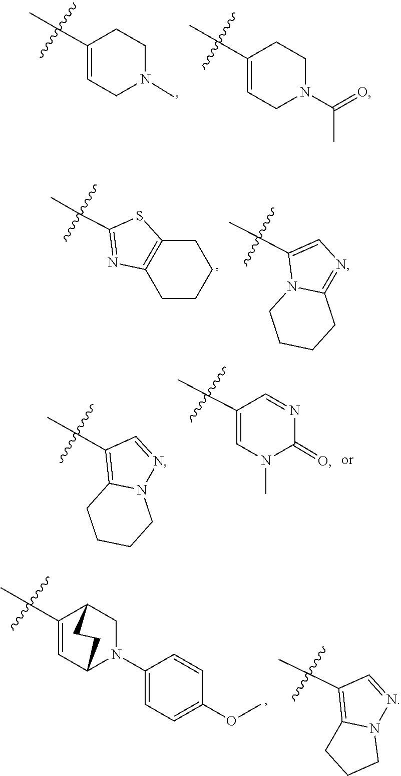

99. The method of claim 96, wherein R_{f} is:

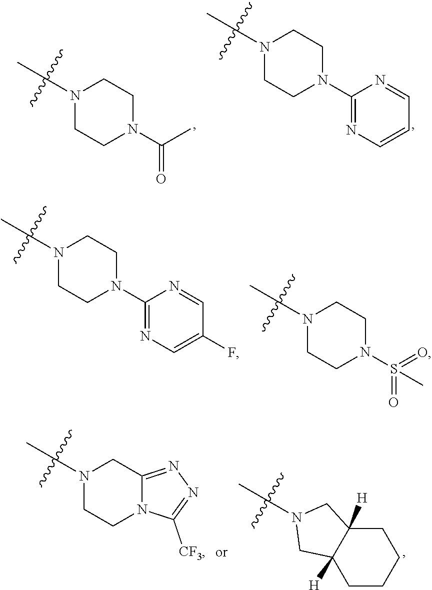

100. The method of claim 83, wherein R_{f} is optionally substituted 5-membered heteroaryl containing 1, 2, or 3 heteroatoms selected from N, O, and S.

101. The method of claim 100, wherein R_{f} is:

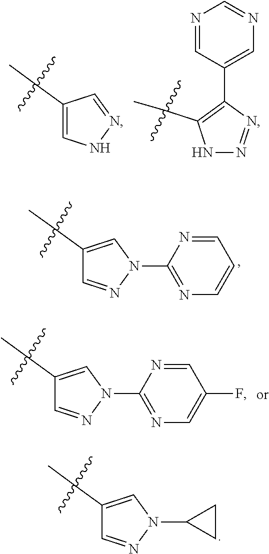 [SMILES: *c1cn[nH]c1 ; *c1[nH]nnc1-c1cncnc1 ; *c1cnn(-c2ncccn2)c1 ; *c1cnn(-c2ncc(F)cn2)c1 ; *c1cnn(C2CC2)c1]

102. The method of any one of claims 23-101, wherein R⁴ is —C(O)R_{b}.

103. The method of claim 102, wherein R⁴ is

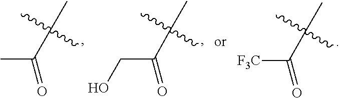

104. The method of claim 103, wherein R⁴ is

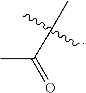 [SMILES: *C(C)=O]

105. The method of any one of claims 23-101, wherein R⁴ is —C(O)NRₐRₐ′.

106. The method of claim 105, wherein R⁴ is:

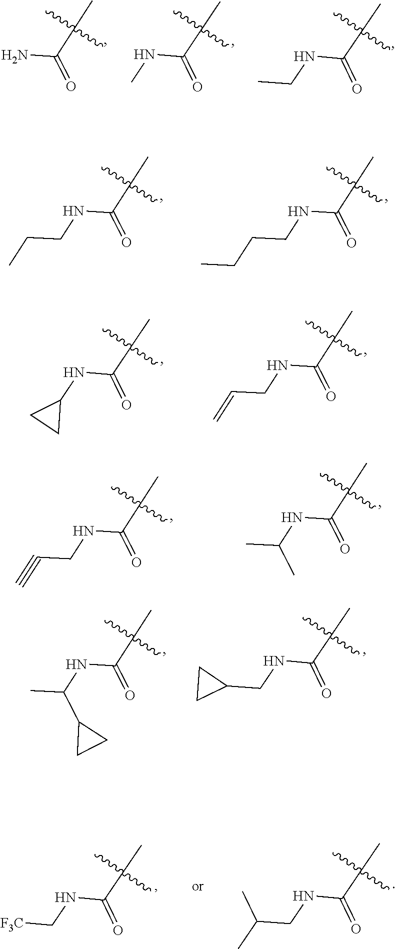

107. The method of claim 106, wherein R⁴ is

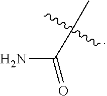 [SMILES: *C(N)=O]

108. The method of any one of claims 23-101, wherein R⁴ is —C(O)OR_{b}.

109. The method of claim 108, wherein R⁴ is —C(O)OCH₃ or —C(O)OH.

110. The method of any one of claims 23-101, wherein R⁴ is optionally substituted C₁-C₆ alkyl.

111. The method of claim 110, wherein R⁴ is

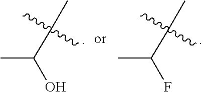

112. The method of any one of claims 23-101, wherein R⁴ is

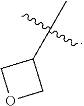 [SMILES: *C1COC1]

113. The method of any one of claims 23-101, wherein R⁴ is cyano.

114. The method of any one of claims 23-101, wherein R⁴ Is halo.

115. The method of any one of claims 23-114, wherein R⁵ is H.

116. The method of any one of claims 23-115, wherein R⁶ is H.

117. The method of any one of claims 23-30, wherein the compound of formula (XIV) is: 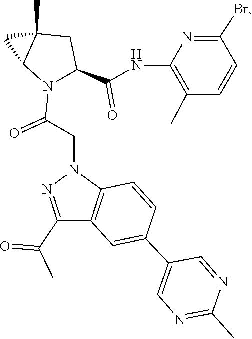 [SMILES: CC(=O)c1nn(CC(=O)N2[C@H](C(=O)Nc3nc(Br)ccc3C)C[C@@]3(C)C[C@@H]23)c2ccc(-c3cnc(C)nc3)cc12]
or a pharmaceutically acceptable salt thereof.

118. The method of any one of claims 23-30, wherein the compound of formula (XIV) is: 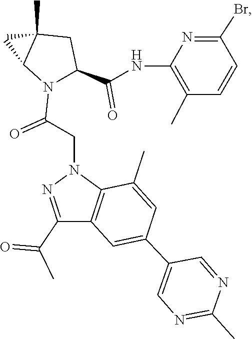 [SMILES: CC(=O)c1nn(CC(=O)N2[C@H](C(=O)Nc3nc(Br)ccc3C)C[C@@]3(C)C[C@@H]23)c2c(C)cc(-c3cnc(C)nc3)cc12]
or a pharmaceutically acceptable salt thereof.

119. The method of any one of claims 23-30, wherein the compound of formula (XIV) is: 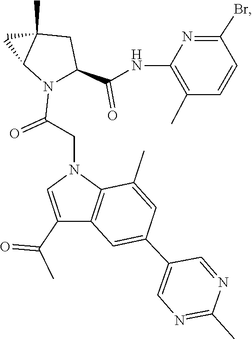 [SMILES: CC(=O)c1cn(CC(=O)N2[C@H](C(=O)Nc3nc(Br)ccc3C)C[C@@]3(C)C[C@@H]23)c2c(C)cc(-c3cnc(C)nc3)cc12]
or a pharmaceutically acceptable salt thereof.

120. The method of any one of claims 23-30, wherein the compound of formula (XIV) is: 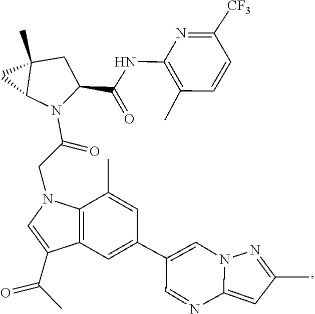 [SMILES: CC(=O)c1cn(CC(=O)N2[C@H](C(=O)Nc3nc(C(F)(F)F)ccc3C)C[C@@]3(C)C[C@@H]23)c2c(C)cc(-c3cnc4cc(C)nn4c3)cc12]
or a pharmaceutically acceptable salt thereof.

121. The method of any one of claims 23-30, wherein the compound of formula (XIV) is: 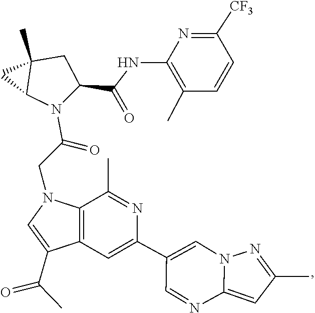 [SMILES: CC(=O)c1cn(CC(=O)N2[C@H](C(=O)Nc3nc(C(F)(F)F)ccc3C)C[C@@]3(C)C[C@@H]23)c2c(C)nc(-c3cnc4cc(C)nn4c3)cc12]
or a pharmaceutically acceptable salt thereof.

122. The method of any one of claims 23-30, wherein the compound of formula (XIV) is: 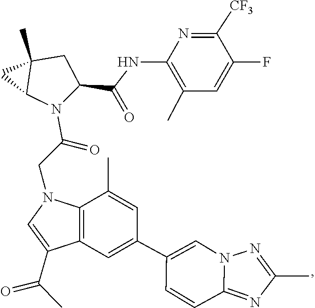 [SMILES: CC(=O)c1cn(CC(=O)N2[C@H](C(=O)Nc3nc(C(F)(F)F)c(F)cc3C)C[C@@]3(C)C[C@@H]23)c2c(C)cc(-c3ccc4nc(C)nn4c3)cc12]
or a pharmaceutically acceptable salt thereof.

123. The method of any one of claims 23-30, wherein the compound of formula (XIV) is: 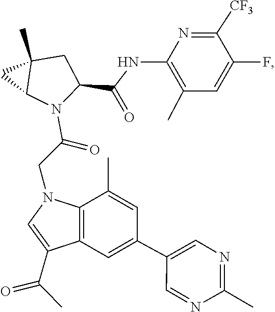 [SMILES: CC(=O)c1cn(CC(=O)N2[C@H](C(=O)Nc3nc(C(F)(F)F)c(F)cc3C)C[C@@]3(C)C[C@@H]23)c2c(C)cc(-c3cnc(C)nc3)cc12]
or a pharmaceutically acceptable salt thereof.
